(19) United States
(12) Patent Application Publication
(10) Pub. No.: US 2011/0201555 A1
(43) Pub. Date: Aug. 18, 2011

(54) NOVEL ESTROGEN RECEPTOR LIGANDS

(76) Inventors: Patrik Rhönnstad, Kungsör (SE); Theresa Apelqvist, Grodinge (SE); Mattias Wennerstål, Hagersten (SE); Aiping Cheng, Huddinge (SE); Sandra Gordon, Mariefred (SE)

(21) Appl. No.: 12/998,065

(22) PCT Filed: Sep. 18, 2009
(86) PCT No.: PCT/EP2009/062144

§ 371 Date: Apr. 11, 2011

(30) Foreign Application Priority Data
Sep. 19, 2008 (GB) 0817221.5

(51) Int. Cl. A61K 31/404; C07D 403/04; C07D 413/04; C07D 209/14; C07D 409/04; A61K 31/422; A61K 31/4155; A61K 31/4178; A61K 31/565; A61K 38/23; A61K 31/59; A61P 19/08; A61P 19/10; A61P 19/00; A61P 3/06; A61P 25/00; A61P 25/28; A61P 25/24; A61P 25/22; A61P 3/04; A61P 25/16; A61P 35/00; A61P 29/00; A61P 37/00

(52) U.S. Cl. 514/11.9; 548/466; 548/247; 548/364.7; 548/312.1; 548/505; 514/414; 514/378; 514/406; 514/397; 514/415; 514/171; 514/167

(57) ABSTRACT

The invention provides a compound of formula (I) or a pharmaceutically acceptable ester, amide, solvate or salt thereof, including a salt of such an ester or amide, and a solvate of such an ester, amide or salt. The invention also provides also provides the use of such compounds in the treatment or prophylaxis of a condition associated with a disease or disorder associated with estrogen receptor activity.

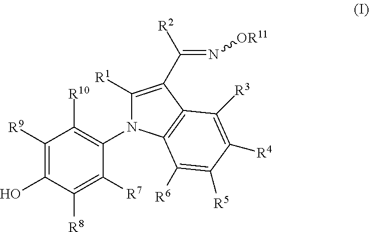

wherein R¹, R², R³, R⁴, R⁵, R⁶, R⁷, R⁸, R⁹ , R¹⁰ and R¹¹ are as defined in the specification.

### FIELD OF INVENTION

This invention relates to compounds which are estrogen receptor ligands and are preferably selective for the estrogen receptor β isoform, to methods of preparing such compounds and to methods for using such compounds in treatment of diseases related to the estrogen receptor such as depressive disorders, anxiety disorders, Alzheimer's disease, cognitive disorders, osteoporosis, elevated blood triglyceride levels, atherosclerosis, endometriosis, urinary incontinence, autoimmune disease, and cancer of the lung, colon, breast, uterus and prostate.

### BACKGROUND OF INVENTION

The estrogen receptor (ER) is a ligand activated mammalian transcription factor involved in the up and down regulation of gene expression. The natural hormone for the estrogen receptor is β-17-estradiol (E2) and closely related metabolites. Binding of estradiol to the estrogen receptor causes a dimerization of the receptor and the dimer in turn binds to estrogen response elements (ERE's) on DNA. The ER/DNA complex recruits other transcription factors responsible for the transcription of DNA downstream from the ERE into mRNA which is eventually translated into protein. Alternatively the interaction of ER with DNA may be indirect through the intermediacy of other transcription factors, most notably fos and jun. Since the expression of a large number of genes is regulated by the estrogen receptor and since the estrogen receptor is expressed in many cell types, modulation of the estrogen receptor through binding of either natural hormones or synthetic ER ligands can have profound effects on the physiology and pathophysiology of the organism.

Historically it has been believed there was only one estrogen receptor. However a second subtype (ER-β) has been discovered. While both the “classical” ER-α and the more recently discovered ER-β are widely distributed in different tissues, they nevertheless display markedly different cell type and tissue distributions. Therefore synthetic ligands which are either ER-α or ER-β selective may preserve the beneficial effects of estrogen while reducing the risk of undesirable side effects.

Estrogens are critical for sexual development in females. In addition, estrogens play an important role in maintaining bone density, regulation of blood lipid levels, and appear to have neuroprotective effects. Consequently decreased estrogen production in post-menopausal women is associated with a number of diseases such as osteoporosis, atherosclerosis, depression and cognitive disorders. Conversely certain types of proliferative diseases such as breast and uterine cancer and endometriosis are stimulated by estrogens and therefore antiestrogens (i.e., estrogen antagonists) have utility in the prevention and treatment of these types of disorders.

The efficacy of the natural estrogen, 17β-estradiol, for the treatment of various forms of depressive illness has also been demonstrated and it has been suggested that the anti-depressant activity of estrogen may be mediated via regulation of tryptophan hydroxylase activity and subsequent serotonin synthesis (See, e.g., Lu N Z, Shlaes T A, Cundlah C, Dziennis S E, Lyle R E, Bethea C L, “Ovarian steroid action on tryptophan hydroxylase protein and serotonin compared to localization of ovarian steroid receptors in midbrain of guinea pigs.” Endocrine 11:257-267, 1999). The pleiotropic nature of natural estrogen precludes its widespread, more chronic use due to the increased risk of proliferative effects on breast, uterine and ovarian tissues. The identification of the estrogen receptor, ERβ, has provided a means by which to identify more selective estrogen agents which have the desired anti-depressant activity in the absence of the proliferative effects which are mediated by ERα. Thus, it has been shown that therapeutic agents having ERβ-selectivity are potentially particularly effective in the treatment of depression.

What is needed in the art are compounds that can produce the same positive responses as estrogen replacement therapy without the negative side effects. Also needed are estrogen-like compounds that exert selective effects on different tissues of the body.

WO 2006/019831 discloses certain indole derivatives having utility in the prevention or treatment of Hepatitis C viral infection. WO 2005/018636 and R. E. Mewshaw et. al., “ERβ ligands. Part 5: Synthesis and structure-activity relationships of a series of 4′-hydroxyphenyl-aryl-carbaldehyde oxime derivatives”, Bioorg. Med. Chem. Lett., 17, 2007, 902-906 both disclose certain indole derivative having estrogen receptor modulator activity, all said indoles being oximes.

The compounds of the present invention are ligands for estrogen receptors and as such may be useful for treatment or prevention of a variety of conditions related to estrogen functioning including bone loss, bone fractures, osteoporosis, cartilage degeneration, endometriosis, uterine fibroid disease, hot flashes, increased levels of LDL cholesterol, cardiovascular disease, impairment of cognitive functioning, age-related mild cognitive impairment, cerebral degenerative disorders, restenosis, gynecomastia, vascular smooth muscle cell proliferation, obesity, incontinence, anxiety, depression, perimenopausal depression, post-partum depression, premenstrual syndrome, manic depression, dementia, obsessive compulsive behavior, attention deficit disorder, attention deficit hyperactivity disorder, sleep disorders, irritability, impulsivity, anger management, hearing disorders, multiple sclerosis, Parkinson's disease, Alzheimer's disease, Huntington's disease, amyotrophic lateral sclerosis, spinal cord injury, stroke, autoimmune disease, inflammation, IBD, IBS, sexual dysfunction, hypertension, retinal degeneration, lung cancer, colon cancer, breast cancer, uterus cancer, prostate cancer and cholangiocarcinoma.

### SUMMARY OF THE INVENTION

This invention provides a compound of formula (I) or a pharmaceutically acceptable ester, amide, solvate or salt thereof, including a salt of such an ester or amide, and a solvate of such an ester, amide or salt,

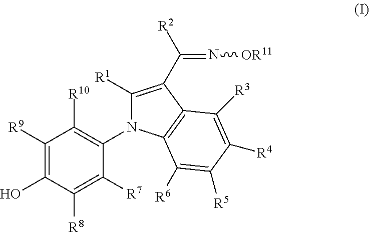

wherein R¹ is selected from the group consisting of C₃₋₈cycloalkyl, phenyl, and 5-10 membered heterocyclyl, wherein said phenyl or heterocyclyl group can be either unsubstituted or substituted with from 1 to 3 substituents, each substituent being selected from the group consisting of OR^{A}, halogen, cyano, nitro, —C(O)C₁₋₄alkyl, C₁₋₆alkyl, C₂₋₆alkenyl, C₂₋₆alkynyl, haloC₁₋₆alkyl, dihaloC₁₋₆alkyl and trihaloC₁₋₆alkyl;

R² is selected from the group consisting of hydrogen, C₁₋₄alkyl, haloC₁₋₄alkyl, dihaloC₁₋₄alkyl, trihaloC₁₋₄alkyl, C₂₋₄alkenyl, and C₂₋₄alkynyl;

each of R³, R⁴, R⁵, R⁶, R⁷, R⁸, R⁹ and R¹⁰ is independently selected from the group consisting of hydrogen, OR^{A}, halogen, cyano, nitro, C₁₋₆alkyl, C₂₋₆alkenyl, C₂₋₆alkynyl, haloC₁₋₆alkyl, dihaloC₁₋₆alkyl and trihaloC₁₋₆alkyl;

each R^{A} is independently selected from the group consisting of hydrogen, C₁₋₆alkyl, C₂₋₆alkenyl, C₂₋₆alkynyl, C₃₋₈cycloalkyl, C₃₋₈cycloalkylC₁₋₆alkyl, C₆₋₁₀aryl and C₆₋₁₀arylC₁₋₆alkyl, each optionally substituted by from 1 to 3 halogen atoms; and

R¹¹ is selected from hydrogen and methyl;

with the proviso that when R¹ is a 5-membered heterocyclyl, and each of R², R³, R⁴, R⁶ and R¹¹ is hydrogen, then R⁵ is not methoxy.

Compounds of the invention have surprisingly been found to be ligands of the estrogen receptor. The compounds accordingly have use in the treatment or prophylaxis of conditions associated with estrogen receptor activity.

### DETAILED DESCRIPTION OF INVENTION

The compounds of the invention may contain chiral (asymmetric) centers or the molecule as a whole may be chiral. The individual stereoisomers (enantiomers and diastereoisomers) and mixtures of these are within the scope of the present invention.

The compounds of the invention contain an oxime group which may be present as the (E) or (Z) oxime isomer. The individual (E) and (Z) oxime isomers and mixtures of these are within the scope of the present invention. Throughout the specification, where the oxime structure includes a wavy line bond, this indicates either that a single isomer is present but the stereochemistry is unknown, or that a mixture of both isomers is present.

The present invention provides compounds that are estrogen receptor ligands. The term “estrogen receptor ligand” as used herein is intended to cover any moiety which binds to an estrogen receptor. The ligand may act as an agonist, a partial agonist, an antagonist or a partial antagonist. The ligand may be ERβ selective or display mixed ERα and ERβ activity. For example, the ligand may act both as an agonist or a partial agonist of ERβ and as an antagonist or a partial antagonist of ERα.

When R¹ represents a heterocyclyl group, this group may be saturated or unsaturated, and may contain one or more, for example two, O, N and/or S atoms. It is preferably 5- or 6-membered. In one aspect R¹ represents a heteroaryl group, for example a 5-membered heteroaryl group. Suitable heterocyclyl groups include furyl, thienyl, pyrrolyl, pyrrolinyl, pyrrolidinyl, oxazolyl, isoxazolyl, thiazolyl, isothiazolyl, imidazolyl, imidazolinyl, imidazolidinyl, pyrazolyl, pyrazolinyl, pyrazolidinyl, pyridyl, morpholinyl, and piperidyl, with isoxazolyl being a particularly preferred heterocyclyl group. Preferred substituents for a heterocyclyl group include 1 to 3, for example 1 or 2, substituents, each substituent being independently selected from the group consisting of OR^{A}, halogen, cyano, —C(O)C₁₋₄alkyl, C₂₋₄alkenyl, C₂₋₄alkynyl, dihaloC₁₋₄alkyl and trihaloC₁₋₄alkyl. Especially preferred substituents are independently selected from halogen, cyano, C₁₋₄alkyl, —C(O)C₁₋₄alkyl, and OR^{A} in which R^{A} preferably represents a hydrogen atom or a C₁₋₄alkyl group. More especially preferred substituents are selected from halogen, cyano and C₁₋₄alkyl (especially methyl or ethyl).

Preferred substituents for a phenyl group R¹ include those mentioned above for a heterocyclyl group R¹. Preferably, a phenyl group R¹ is unsubstituted.

Unless otherwise stated, each R^{A} is preferably independently selected from the group consisting of hydrogen, C₁₋₄alkyl, C₂₋₄alkenyl, C₂₋₄alkynyl, C₃₋₆cycloalkyl, phenyl and benzyl. Preferably each R^{A} independently represents hydrogen or C₁₋₄alkyl, especially methyl.

Preferably R¹ is selected from the group consisting of phenyl and 5-6 membered heterocyclyl, wherein said phenyl or heterocyclyl group can either be unsubstituted or substituted as above. More preferably, R¹ is selected from the group consisting of phenyl and 5- membered heterocyclyl, wherein said phenyl or heterocyclyl group can either be unsubstituted or substituted as above. In one embodiment, R¹ represents a phenyl group or a 5-membered heterocyclyl group containing one or two heteroatoms selected from O, N and/or S, wherein said phenyl or 5-membered heterocyclyl group is optionally substituted by 1, 2 or 3 substituents selected from halogen, cyano and C₁₋₄alkyl. In a further embodiment, R¹ represents an unsubstituted phenyl group or a 5-membered heterocyclyl group containing one or two heteroatoms selected from O, N and/or S, said heterocyclyl group being optionally substituted by 1, 2 or 3 substituents selected from cyano, methyl and ethyl.

Preferably, R² is selected from the group consisting of hydrogen, C₁₋₄alkyl, C₂₋₄alkenyl, C₂₋₄alkynyl and trihaloC₁₋₄alkyl. In one aspect of the invention, R² is selected from the group selected from hydrogen, C₁₋₄alkyl, especially methyl, and trihaloC₁₋₄alkyl, especially trifluoromethyl. In another aspect of the invention, R² is selected from the group consisting of hydrogen, C₁₋₄alkyl, C₂₋₄alkenyl, and C₂₋₄alkynyl More preferably, R² is hydrogen or C₁₋₄alkyl, especially methyl. Most preferably, R² is hydrogen.

Preferably each of R³, R⁴, R⁵, R⁶, R⁷, R⁸, R⁹ and R¹⁰ is independently selected from the group consisting of hydrogen, OR^{A}, halogen, cyano, C₁₋₄alkyl, for example methyl, haloC₁₋₄alkyl, for example chloro- or fluoro-methyl, dihaloC₁₋₄alkyl, for example dichloro- or difluoromethyl, and trihaloC₁₋₄alkyl, for example trichloro- or trifluoromethyl. In one aspect of the invention, each of R³, R⁴, R⁵, R⁶, R⁷, R⁸, R⁹ and R¹⁰ is independently selected from the group consisting of hydrogen, halogen, cyano, C₁₋₄alkyl, for example methyl, haloC₁₋₄alkyl, for example chloro- or fluoro-methyl, dihaloC₁₋₄alkyl, for example dichloro- or difluoromethyl, and trihaloC₁₋₄alkyl, for example trichloro- or trifluoromethyl. Preferably each of R³, R⁴, R⁵, R⁶, R⁷, R⁸, R⁹ and R¹⁰ is independently selected from the group consisting of hydrogen, OH, halogen, cyano, methyl, or trifluoromethyl. More preferably, each of R³, R⁴, R⁵, R⁶, R⁷, R⁸, R⁹ and R¹⁰ is independently selected from the group consisting of hydrogen, halogen, methyl or trifluoromethyl. Most preferably each of R³, R⁴, R⁵, R⁶, R⁷, R⁸, R⁹ and R¹⁰ independently represents hydrogen and/or halogen, for example chlorine or fluorine, and especially fluorine.

In a further embodiment of the invention, each of R³, R⁴, R⁶, R⁷, R⁸, R⁹ and R¹⁰ is independently selected from the group consisting of hydrogen, OR^{A}, halogen, cyano, nitro, C₁₋₆alkyl, C₂₋₆alkenyl, C₂₋₆alkynyl, haloC₁₋₆alkyl, dihaloC₁₋₆alkyl and trihaloC₁₋₆alkyl; and R⁵ is selected from the group consisting of hydrogen, OH, halogen, cyano, nitro, C₁₋₆alkyl, C₂₋₆alkenyl, C₂₋₆alkynyl, haloC₁₋₆alkyl, dihaloC₁₋₆alkyl, and trihaloC₁₋₆alkyl. In this embodiment, R⁵ is preferably selected from the group consisting of hydrogen, halogen, cyano, nitro, C₁₋₆alkyl, C₂₋₆alkenyl, C₂₋₆alkynyl, haloC₁₋₆alkyl, dihaloC₁₋₆alkyl and trihaloC₁₋₆alkyl.

Preferably, R¹¹ is hydrogen.

Accordingly, in one preferred group of compounds of the invention, R¹ is selected from the group consisting of phenyl, and 5-6 membered heterocyclyl, wherein said phenyl or heterocyclyl group may be either unsubstituted or substituted as above; more preferably, R¹ is selected from the group consisting of phenyl, and 5- membered heterocyclyl, wherein said phenyl or heterocyclyl group can either be unsubstituted or substituted as above;

R² is selected from the group consisting of hydrogen, C₁₋₄alkyl, C₂₋₄alkenyl, and C₂₋₄alkynyl;

each of R³, R⁴, R⁵, R⁶, R⁷, R⁸, R⁹ and R¹⁰ is independently selected from the group consisting of hydrogen, OR^{A}, halogen, cyano, C₁₋₄alkyl, haloC₁₋₄alkyl, dihaloC₁₋₄alkyl, and trihaloC₁₋₄alkyl, especially hydrogen, OH, halogen, cyano, methyl, or trifluoromethyl; especially, each of R³, R⁴, R⁵, R⁶, R⁷, R⁸, R⁹ and R¹⁰ independently represents hydrogen and/or halogen, especially fluorine;

each R^{A} is independently selected from the group consisting of hydrogen, C₁₋₄alkyl, C₂₋₄alkenyl, C₂₋₄alkynyl, C₃₋₆cycloalkyl, phenyl and benzyl, especially hydrogen and C₁₋₄alkyl, especially methyl; and R¹¹ is hydrogen.

In another preferred group of compounds of the invention, R¹ represents an unsubstituted phenyl group or a 5-membered heterocyclyl group containing one or two heteroatoms selected from O, N and/or S, said heterocyclyl group being optionally substituted by 1, 2 or 3 substituents selected from cyano, methyl or ethyl; R² is selected from the group selected from hydrogen, methyl and trifluoromethyl; and each of R³, R⁴, R⁵, R⁶, R⁷, R⁸, R⁹ and R¹⁰ is independently selected from the group consisting of hydrogen, halogen, methyl or trifluoromethyl.

Compounds of the formula (I) include, but are not limited to, the compounds specifically named in the Examples herein. In the Examples, the compound names were generated in accordance with IUPAC by the ACD Labs 8.0/name program, version 8.05 and/or with ISIS DRAW Autonom 2000.

Depending upon the substituents present in compounds of the formula I, the compounds may form esters, amides and/or salts. Salts and solvates of compounds of formula (I) which are suitable for use in medicine are those wherein a counterion or associated solvent is pharmaceutically acceptable. However, salts and solvates having non-pharmaceutically acceptable counterions or associated solvents are within the scope of the present invention, for example, for use as intermediates in the preparation of the compounds of formula (I) and their pharmaceutically acceptable salts, solvates and physiologically functional derivatives. By the term “physiologically functional derivative” is meant a chemical derivative of a compound of formula (I) having the same physiological function as the free compound of formula (I), for example, by being convertible in the body thereto. Esters and amides are examples of physiologically functional derivatives.

Suitable salts according to the invention include those formed with organic or inorganic acids or bases. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄)-alkyl- or aryl-sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Other acids such as oxalic, while not in themselves pharmaceutically acceptable, may be useful as intermediates in obtaining the compounds of the invention and their pharmaceutical acceptable acid addition salts.

Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. Corresponding internal salts may furthermore be formed.

Compounds of formula (I) may have an appropriate group converted to an ester or an amide. Thus typical ester and amide groups formed from an —OH group in the compound of the formula (I) include —O.CO.R^{B}; —NR^{B}.CO.R^{B}, —O.SO₂R^{B}, and —NR^{B}.SO₂R^{B}, where each R^{B} is independently selected from the group consisting of hydrogen, C₁₋₆alkyl, C₂₋₆alkenyl, C₂₋₆alkynyl, C₃₋₈cycloalkyl, C₃₋₈cycloalkylC₁₋₆alkyl, C₆₋₁₀aryl and C₆₋₁₀arylC₁₋₆alkyl, each optionally substituted by from 1 to 3 halogen atoms. Preferred R^{B} groups are hydrogen and C₁₋₄alkyl. For example, in the compounds of formula (I) above, the hydroxy group on the benzene ring may be converted to an ester group, and/or the hydroxy group of the oxime may be converted to an ester group.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

A compound which, upon administration to the recipient, is capable of being converted into a compound of formula (I) as described above, or an active metabolite or residue thereof, is known as a “prodrug”. A prodrug may, for example, be converted within the body, e. g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series (1976); “Design of Prodrugs” ed. H. Bundgaard, Elsevier, 1985; and in Edward B. Roche, ed., Bioreversible Carriers in Drug Design, American Pharmaceutical Association and Pergamon Press, 1987, which are incorporated herein by reference.

The following definitions apply to the terms as used throughout this specification, unless otherwise limited in specific instances.

As used herein, the term “alkyl” means both straight and branched chain saturated hydrocarbon groups. Examples of alkyl groups include methyl, ethyl, n-propyl, iso-propyl, n-butyl, t-butyl, i-butyl, sec-butyl, pentyl and hexyl groups. Among unbranched alkyl groups, there are preferred methyl, ethyl, n-propyl, iso-propyl, n-butyl groups. Among branched alkyl groups, there may be mentioned t-butyl, i-butyl, 1-ethylpropyl and 1-ethylbutyl groups.

As used herein, the term “alkoxy” means the group O-alkyl, where “alkyl” is used as described above. Examples of alkoxy groups include methoxy and ethoxy groups. Other examples include propoxy and butoxy.

As used herein, the term “alkenyl” means both straight and branched chain unsaturated hydrocarbon groups with at least one carbon carbon double bond. Examples of alkenyl groups include ethenyl, propenyl, butenyl, pentenyl and hexenyl. Preferred alkenyl groups include ethenyl, 1- propenyl and 2-propenyl.

As used herein, the term “alkynyl” means both straight and branched chain unsaturated hydrocarbon groups with at least one carbon carbon triple bond. Examples of alkynyl groups include ethynyl, propynyl, butynyl, pentynyl and hexynyl. Preferred alkynyl groups include ethynyl 1- propynyl and 2-propynyl.

As used herein, the term “cycloalkyl” means a saturated group in a ring system. A cycloalkyl group can be monocyclic or bicyclic. A bicyclic group may, for example, be fused or bridged. Examples of monocyclic cycloalkyl groups include cyclopropyl, cyclobutyl and cyclopentyl. Other examples of monocyclic cycloalkyl groups are cyclohexyl, cycloheptyl and cyclooctyl. Examples of bicyclic cycloalkyl groups include bicyclo[2.2.1]hept-2-yl. Preferably, the cycloalkyl group is monocyclic.

As used herein, the term “aryl” means a monocyclic or bicyclic aromatic carbocyclic group. Examples of aryl groups include phenyl and naphthyl. A naphthyl group may be attached through the 1 or the 2 position. In a bicyclic aromatic group, one of the rings may, for example, be partially saturated. Examples of such groups include indanyl and tetrahydronaphthyl. Specifically, the term C₅₋₁₀ aryl is used herein to mean a group comprising from 5 to 10 carbon atoms in a monocyclic or bicyclic aromatic group. A particularly preferred C₅₋₁₀ aryl group is phenyl.

As used herein, the term “halogen” means fluorine, chlorine, bromine or iodine. Fluorine, chlorine and bromine are particularly preferred.

As used herein, the term “haloalkyl” means an alkyl group having a halogen substituent, the terms “alkyl” and “halogen” being understood to have the meanings outlined above. Similarly, the term “dihaloalkyl” means an alkyl group having two halogen substituents and the term “trihaloalkyl” means an alkyl group having three halogen substituents. Examples of haloalkyl groups include fluoromethyl, chloromethyl, bromomethyl, fluoromethyl, fluoropropyl and fluorobutyl groups; examples of dihaloalkyl groups include difluoromethyl and difluoroethyl groups; examples of triihaloalkyl groups include trifluoromethyl and trifluoroethyl groups.

As used herein, the term “heterocyclyl” means an aromatic or a non-aromatic cyclic group of carbon atoms wherein from one to three of the carbon atoms is/are replaced by one or more heteroatoms independently selected from nitrogen, oxygen or sulfur. A heterocyclyl group may, for example, be monocyclic or bicyclic. In a bicyclic heterocyclyl group there may be one or more heteroatoms in each ring, or only in one of the rings. A heteroatom is preferably O, N or S, for example O or N. Heterocyclyl groups containing a suitable nitrogen atom include the corresponding N-oxides.

Examples of monocyclic non-aromatic heterocyclyl groups (also referred to as monocyclic heterocycloalkyl rings) include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl.

Examples of bicyclic heterocyclyl groups in which one of the rings is non-aromatic include dihydrobenzofuranyl, indanyl, indolinyl, isoindolinyl, tetrahydroisoquinolinyl, tetrahydroquinolyl and benzoazepanyl.

Examples of monocyclic aromatic heterocyclyl groups (also referred to as monocyclic heteroaryl groups) include furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl and pyrimidinyl.

Examples of bicyclic aromatic heterocyclyl groups (also referred to as bicyclic heteroaryl groups) include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridiyl, pyridopyrimidinyl, isoquinolinyl and benzodroxazole.

Examples of preferred heterocyclyl groups include piperidinyl, tetrahydrofuranyl, tetrahydropyranyl, pyridyl, pyrimidinyl and indolyl. Preferred heterocyclyl groups also include thiophenyl, thiazolyl, furanyl, pyrazolyl, pyrrolyl, isoxazolyl and imidazolyl.

As used herein the term “cycloalkylalkyl” means a group cycloalkyl-alkyl-attached through the alkyl group, “cycloalkyl” and “alkyl” being understood to have the meanings outlined above.

As mentioned above, the compounds of the invention have activity as estrogen receptor ligands. The compounds of the invention have activity as estrogen receptor modulators, and may be agonists, partial agonists, antagonists, or partial antagonists of the estrogen receptor. Particularly preferred compounds of the invention have activity as an agonist or a partial agonist of ERβ. Preferred compounds of this type are selective agonists of the estrogen receptor-beta (ERβ).

The compounds of the invention may thus be used in the treatment of diseases or disorders associated with estrogen receptor activity. In particular, the compounds of the invention that are agonists or partial agonists of the estrogen receptor may be used in the treatment of diseases or disorders for which selective agonists or partial agonists of the estrogen receptor are indicated. The compounds of the invention that are antagonists or partial antagonists of the estrogen receptor may be used in the treatment of diseases or disorders for which selective antagonists or partial antagonists of the estrogen receptor are indicated.

Clinical conditions for which an agonist or partial agonist is indicated include, but are not limited to, bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, inflammation, IBD, IBS, sexual dysfunction, hypertension, retinal degeneration, and lung, colon, breast, uterus, and prostate cancer, and/or disorders related to estrogen functioning.

The compounds of the invention find particular application in the treatment or prophylaxis of the following: bone loss, bone fractures, osteoporosis, cartilage degeneration, endometriosis, uterine fibroid disease, hot flashes, increased levels of LDL cholesterol, cardiovascular disease, impairment of cognitive functioning, age-related mild cognitive impairment, cerebral degenerative disorders, restenosis, gynecomastia, vascular smooth muscle cell proliferation, obesity, incontinence, anxiety, depression, perimenopausal depression, post-partum depression, premenstrual syndrome, manic depression, dementia, obsessive compulsive behavior, attention deficit disorder, attention deficit hyperactivity disorder, sleep disorders, irritability, impulsivity, anger management, hearing disorders, multiple sclerosis, Parkinson's disease, Alzheimer's disease, Huntington's disease, amyotrophic lateral sclerosis, spinal cord injury, stroke, autoimmune disease, inflammation, IBD, IBS, sexual dysfunction, hypertension, retinal degeneration, lung cancer, colon cancer, breast cancer, uterus cancer, prostate cancer and the bile duct cancer form named cholangiocarcinoma.

In one embodiment of the invention, the present compounds finds particular application in the treatment or prophylaxis of depression, perimenopausal depression, post-partum depression, premenstrual syndrome and manic depression.

The treatment or prophylaxis of hot flashes (or hot flushes) in males, is preferable for patients that has had an androgen ablation for treatment of prostate cancer.

The phrase “depression” includes but is not limited to, major depressive disorder, dysthymic disorder, bipolar disorder, cyclothymic disorder, mood disorder due to a general medical condition, substance-induced mood misorder, seasonal affective disorder (SAD), postpartum depression and premenstrual dysphoric disorder.

The invention also provides a method for the treatment or prophylaxis of a condition in a mammal mediated by an estrogen receptor, which comprises administering to the mammal a therapeutically effective amount of a compound according to the invention. Clinical conditions mediated by an estrogen receptor that may be treated by the method of the invention are preferably those described above.

The invention also provides the use of a compound according to the invention, for the manufacture of a medicament for the treatment or prophylaxis of a condition mediated by an estrogen receptor. Clinical conditions mediated by an estrogen receptor that may be treated by the method of the invention are preferably those described above.

The amount of active ingredient which is required to achieve a therapeutic effect will, of course, vary with the particular compound, the route of administration, the subject under treatment, including the type, species, age, weight, sex, and medical condition of the subject and the renal and hepatic function of the subject, and the particular disorder or disease being treated, as well as its severity. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 mg per kg of body weight per day (mg/kg/day) to 10 mg/kg/day, and most preferably 0.1 to 5.0 mg/kg/day, for adult humans. For oral administration, the compositions are preferably provided in the form of tablets or other forms of presentation provided in discrete units containing 0.01, 0.05, 0.1, 0.5, 1.0, 2.5, 5.0, 10.0, 15.0, 25.0, 50.0, 100, and 500 milligrams of the active ingredient for the symptomatic adjustment of the dosage to the patient to be treated. A medicament typically contains from about 0.01 mg to about 500 mg of the active ingredient, preferably from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

While it is possible for the active ingredient to be administered alone, it is preferable for it to be present in a pharmaceutical formulation or composition. Accordingly, the invention provides a pharmaceutical formulation comprising a compound according to the invention, and a pharmaceutically acceptable diluent, excipient or carrier (collectively referred to herein as “carrier” materials). Pharmaceutical compositions of the invention may take the form of a pharmaceutical formulation as described below.

The pharmaceutical formulations according to the invention include those suitable for oral, parenteral (including subcutaneous, intradermal, intramuscular, intravenous [bolus or infusion], and intraarticular), inhalation (including fine particle dusts or mists which may be generated by means of various types of metered does pressurized aerosols), nebulizers or insufflators, rectal, intraperitoneal and topical (including dermal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets, pills or tablets each containing a predetermined amount of the active ingredient; as a powder or granules; as a solution or a suspension in an aqueous liquid or a non-aqueous liquid, for example as elixirs, tinctures, suspensions or syrups; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compounds can also be administered liposomally.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate, calcium sulfate, sorbitol, glucose and/or lactose and/or other excipients, binders, extenders, disintegrants, diluents and lubricants such as those known in the art. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Disintegrators include without limitation starch, methylcellulose, agar, bentonite, xanthan gum and the like. The compounds of formula (I) can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use. Lubricants used in these dosage forms include sodium oleate, sodium stearate, magnesium stearate, sodium benzoate, sodium acetate, sodium chloride and the like. For oral administration in liquid form, the oral drug components can be combined with any oral, non-toxic, pharmaceutically acceptable inert carrier such as ethanol, glycerol, water, and the like.

The compounds of the present invention can also be administered in the form of liposome delivery systems, such as small unilamellar vesicles, large unilamellar vesicles and multilamellar vesicles. Liposomes can be formed from a variety of phospholipids, 1,2-dipalmitoylphosphatidylcholine, phosphatidyl ethanolamine (cephaline), or phosphatidylcholine (lecithin).

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for nasal, aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

Whilst a compound of the invention may be used as the sole active ingredient in a medicament, it is also possible for the compound to be used in combination with one or more further active agents. Such further active agents may be further compounds according to the invention, or they may be different therapeutic agents, for example an antidepressant, an anxiolytic, an anti-psychotic, or an agent useful in the prevention or treatment of osteoporosis, an agent useful in the prevention or treatment of cancer or other pharmaceutically active material. For example, the compounds of the instant invention may be effectively administered in combination with effective amounts of other agents such as an antidepressant, an anxiolytic, an anti-psychotic, an organic bisphosphonate or a cathepsin K inhibitor. Nonlimiting examples of antidepressants include noradrenaline reuptake inhibitors (NRI), selective serotonin reuptake inhibitors, monoamine oxidase inhibitors, tricyclic antidepressants (TCA), dopamine reuptake inhibitors (DRI), opioids, selective seretonic reuptake enhancers, tetracyclic antidepressants, reversible inhibitors of monoamine oxidase, melatonin agonists, serotonin and noradrenaline reuptake inhibitors (SNRI), corticotropin releasing factor antagonists, α-adrenoreceptor antagonists, 5HT1α receptor agonists and antagonists, lithium and atypical anti-psychotics. Examples of antidepressants of the SSRI class include Fluoxetine and Sertraline; examples of antidepressants of the SNRI class Venlafaxine, Citalopram, Paroxetine, Escitalopram, Fluvoxamine; examples of antidepressants of the SNRI class include Duloxetine; examples of antidepressants of the DRI and NRI classes include Bupropion; examples of antidepressants of the TCA class include Amitriptyline and Dothiepin (Dosulepin). Examples of atypical antipsychotics include: Clozapine, Olanzapine, Risperidone, Quetiapine, Ziprasidone and Dopamine partial agonists. Nonlimiting examples of anxiolytics include benzodiazepines and non-benzodiazapines. Examples of benzodiazapines include lorazepam, alprazolam, and diazepam. Examples of non-benzodiazapines include Buspirone (Buspar®), barbiturates and meprobamate. One or more of those further anti-depressants may be used in combination.

Examples of anti-cancer agents include tamoxifene or an aromatase inhibitor, used in treatment of breast cancer.

In the event that hot flashes are induced by a particular treatment, a compound of the invention may be used in combination therapy with the agent of such treatment. Nonlimiting examples of such combination treatment therapies include: a compound of the invention in combination with tamoxifene treatment of breast cancer, a compound of the invention in combination with aromatase inhibitor treatment of breast cancer, or a compound of the invention in combination with raloxifene treatment of osteoporosis.

Nonlimiting examples of said organic bisphosphonates include adendronate, clodronate, etidronate, ibandronate, incadronate, minodronate, neridronate, risedronate, piridronate, pamidronate, tiludronate, zoledronate, pharmaceutically acceptable salts or esters thereof, and mixtures thereof. Preferred organic biphosphonates include alendronate and pharmaceutically acceptable salts and mixtures thereof. Most preferred is alendronate monosodium trihydrate.

The precise dosage of the bisphosphonate will vary with the dosing schedule, the oral potency of the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. An appropriate amount can be determined by routine experimentation from animal models and human clinical studies. Generally, an appropriate amount of bisphosphonate is chosen to obtain a bone resorption inhibiting effect, i.e. a bone resorption inhibiting amount of the bisphonsphonate is administered. For humans, an effective oral dose of bisphosphonate is typically from about 1.5 to about 6000 μg/kg of body weight and preferably about 10 to about 2000 μg/kg of body weight.

For human oral compositions comprising alendronate, pharmaceutically acceptable salts thereof, or pharmaceutically acceptable derivatives thereof, a unit dosage typically comprises from about 8.75 mg to about 140 mg of the alendronate compound, on an alendronic acid active weight basis, i.e. on the basis of the corresponding acid.

The compounds of the present invention can be used in combination with other agents useful for treating estrogen-mediated conditions. The individual components of such combinations can be administered separately at different times during the course of therapy or concurrently in divided or single combination forms. The present invention is therefore to be understood as embracing all such regimes of simultaneous or alternating treatment and the term “administering” is to be interpreted accordingly. It will be understood that the scope of combinations of the compounds of this invention with other agents useful for treating estrogen-mediated conditions includes in principle any combination with any pharmaceutical composition useful for treating disorders related to estrogen functioning.

The above other therapeutic agents, when employed in combination with the compounds of the present invention, may be used, for example, in those amounts indicated in the Physicians' Desk Reference (PDR) or as otherwise determined by one of ordinary skill in the art.

Where the compounds of the invention are utilized in combination with one or more other therapeutic agent(s), either concurrently or sequentially, the following combination ratios and dosage ranges are preferred:

When combined with an antidepressant, an anxiolytic, an anti-psychotic, an organic bisphosphonate or a cathepsin K inhibitor, the compounds of formula (I) may be employed in a weight ratio to the additional agent within the range from about 10:1 to about 1:10.

The compounds of the invention as described above also find use, optionally in labelled form, as a diagnostic agent for the diagnosis of conditions associated with malfunction of the estrogen receptor. For example, such a compound may be radioactively labelled.

The compounds of the invention as described above, optionally in labelled form, also find use as a reference compound in methods of discovering other agonists, partial agonists, antagonists or partial antagonists of the estrogen receptor. Thus, the invention provides a method of discovering a ligand of the estrogen receptor which comprises use of a compound of the invention or a compound of the invention in labelled form, as a reference compound. For example, such a method may involve a competitive binding experiment in which binding of a compound of the invention to the estrogen receptor is reduced by the presence of a further compound which has estrogen receptor-binding characteristics, for example stronger estrogen receptor-binding characteristics than the compound of the invention in question.

Numerous synthetic routes to the compounds of the present invention can be devised by any person skilled in the art and the possible synthetic routes described below do not limit the invention. Many methods exist in the literature for the synthesis of indoles, for example: Indoles Part One, W. J. Houlihan (ed.), 1972; Indoles, Sundberg, R. J., 1996; Heterocyclic Chemistry, Joule, J. A.; Mills, K. 2000; Chem. Rev., 2005, 105, 2873-2920; Org. Lett., 2006, 8, 5919-5922; and Bioorg. Med. Chem. Lett., 2007, 17, 902-906. A number of possible synthetic routes are shown schematically below. Where appropriate, any initially produced compound according to the invention can be converted into another compound according to the invention by known methods.

General Method I

The following general method can be used to prepare compounds of formula (I) wherein R² is hydrogen.

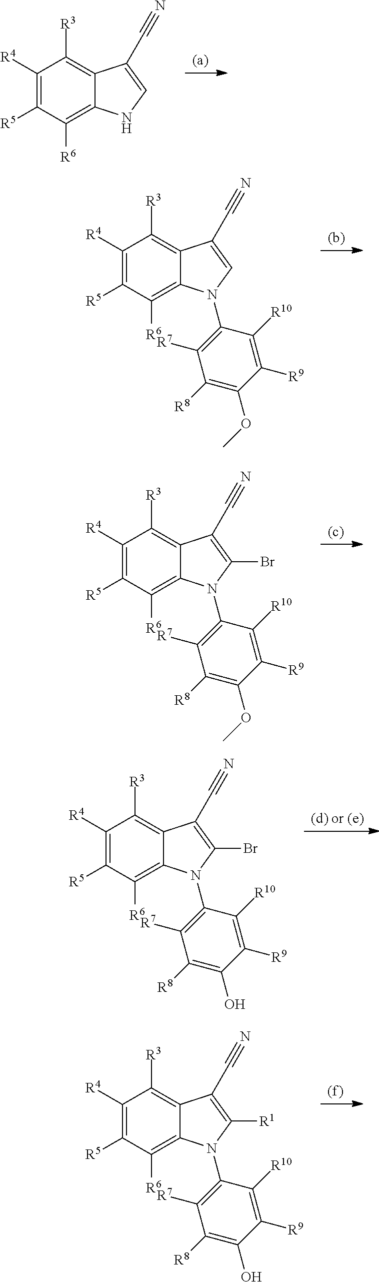

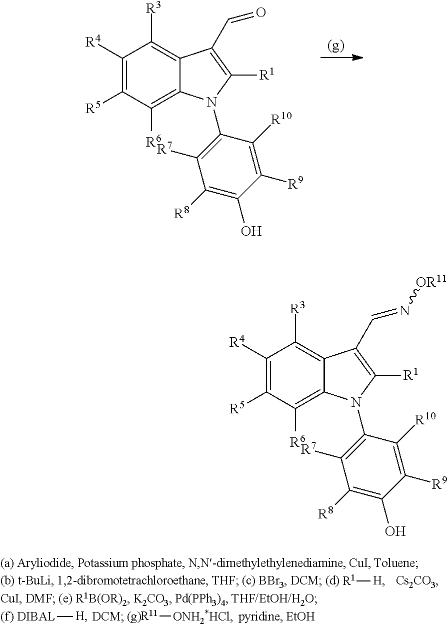

General Method I as shown in the reaction scheme above was used for the synthesis of the following Examples: 1, 2, 5, 6, 7, 8, 10, 11, 12, 13, 14, 15, 16, 17, 18, 26, 27, 28, 29, 30, 38, 40, 41, 42 and 43. Full experimental details of the individual steps of the general method applicable for the synthesis of the final compounds of those Examples are described in Examples 1 (steps (a)-(f) of which provide experimental details for steps (a)-(d), (f) and (g) of General Method I shown in the scheme above) and 2 (steps (a)-(c) of which provide experimental details for steps (e)-(g) of General Method I shown in the scheme above).

General Method II

The following general method can be used to prepare compounds of formula (I) wherein R² is hydrogen.

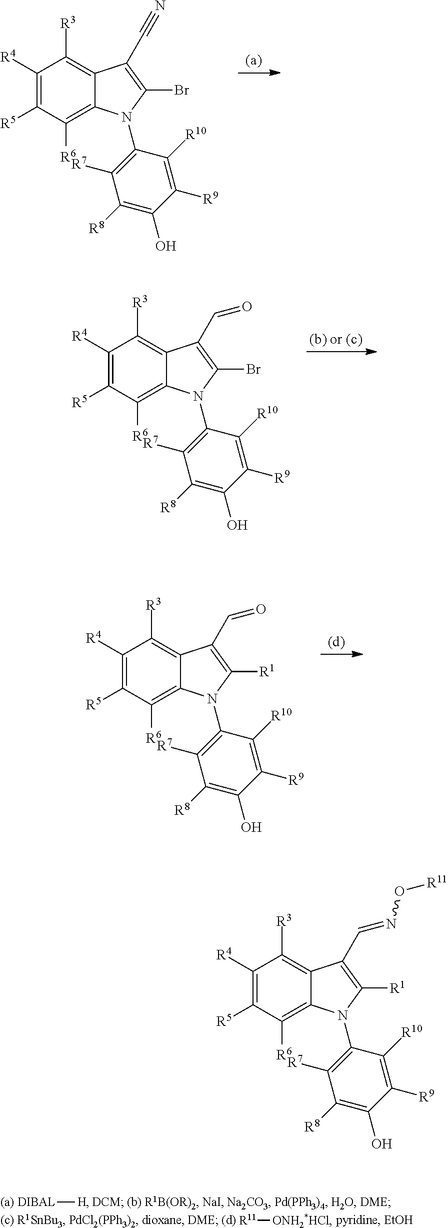

General Method II as shown in the reaction scheme above was used for the synthesis of Examples 3, 4 and 35, and full experimental details of the individual steps of the general method are described in those Examples.

General Method III

The following general method can be used to prepare compounds of formula (I) wherein R² is hydrogen and R¹¹ is hydrogen.

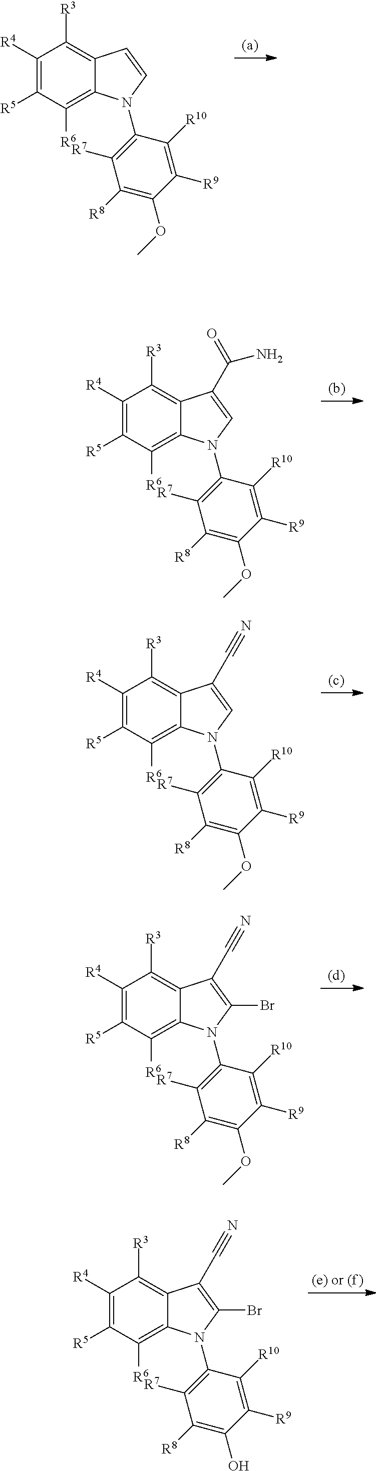

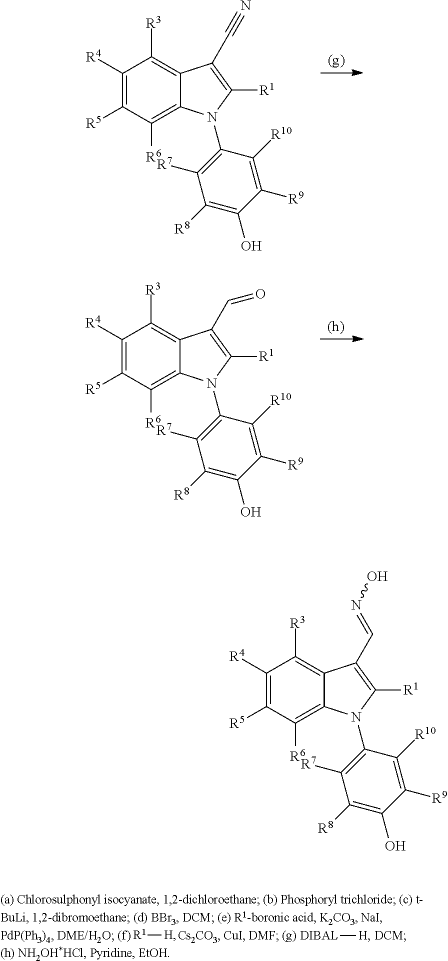

General Method III as shown in the reaction scheme above was used for the synthesis of the following Examples: 19, 20, 21, 22, 23, 24, 25, 31, 36, 37, 39, 44, 45, 46, 47, 48 and 49. Full experimental details of the individual steps of the general method applicable for the synthesis of the final compounds of those Examples are described in Example 31.

General Method IV

The following general method can be used to prepare compounds of formula (1) wherein R² is methyl and R¹¹ is hydrogen.

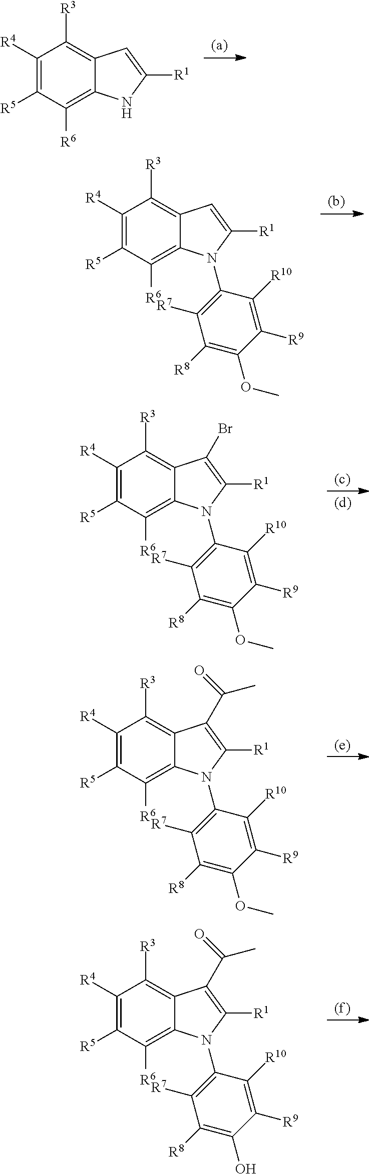

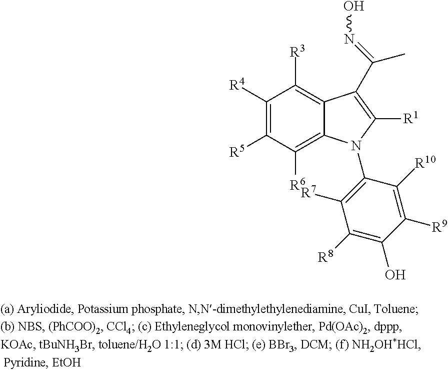

General Method IV as shown in the reaction scheme above was used for the synthesis of Examples 9 and 34, and full experimental details of the individual steps of the general method are described in those Examples.

General Method V

The following general method can be used to prepare compounds of formula (I) wherein R² is trifluoromethyl and R¹¹ is hydrogen.

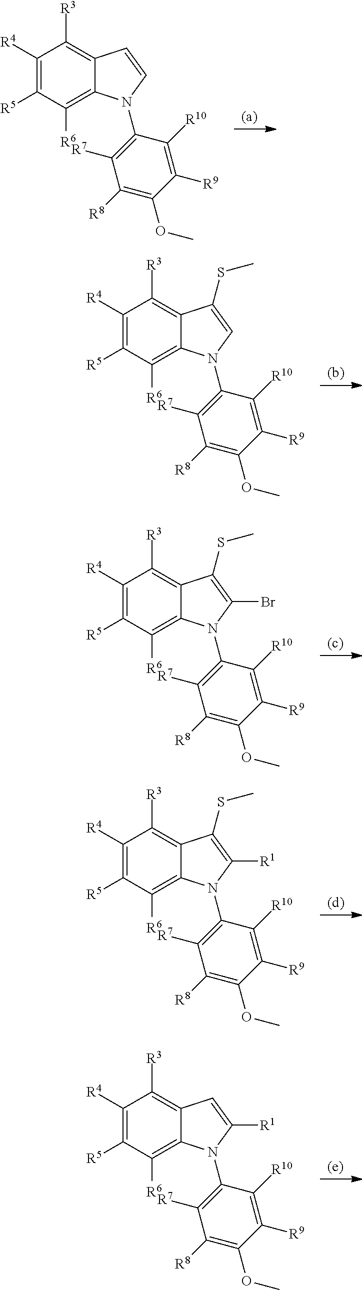

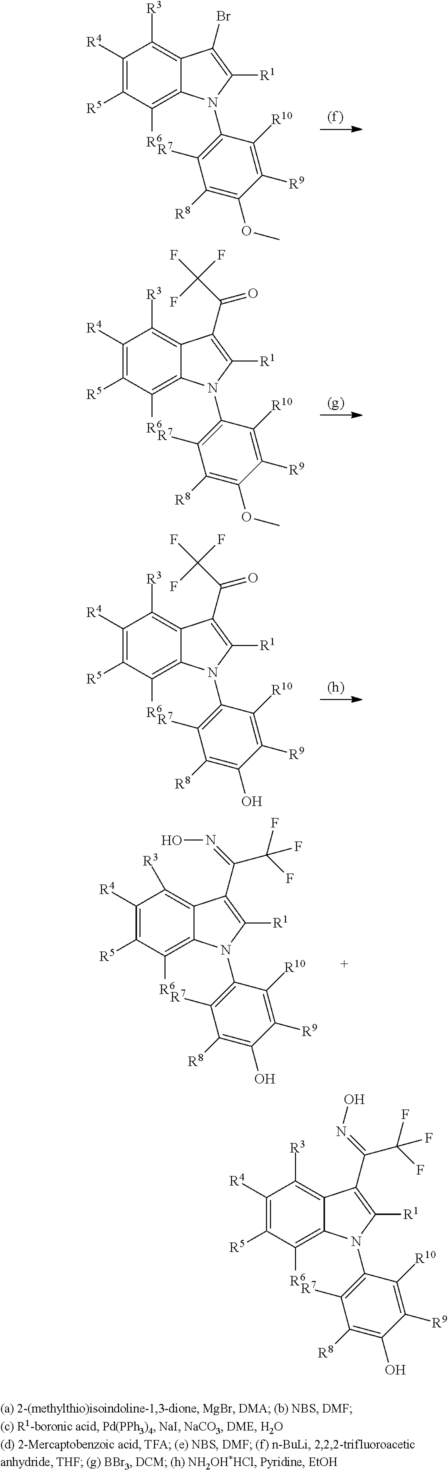

General Method V as shown in the reaction scheme above was used for the synthesis of Examples 32 and 33, and full experimental details of the individual steps of the general method are described in those Examples.

The following Examples illustrate the invention.

### EXAMPLE 1

### 1-(4-Hydroxy-phenyl)-2-pyrrol-1-yl-1H-indole-3-carbaldehyde oxime (E1)

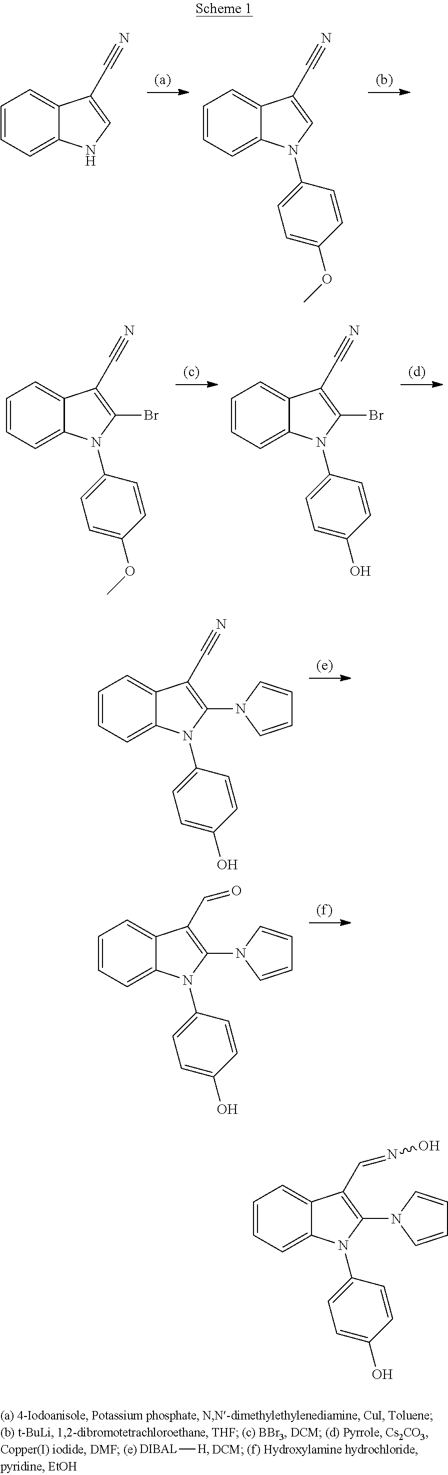

Step (a): 1 eq 3-Cyanoindole, 2 eq 4-Iodoanisole, 2.1 eq Potassium phosphate, 4.5 eq N,N′-dimethylethylenediamine and 0.2 eq Copper(I)iodide were mixed in oven-dried vial and Toluene added. The mixture was stirred under N₂-atmosphere at 110° C. over night. The reaction mixture was cooled to rt, filtered and evaporated in vacuo. The crude product was purified on silica column using 4:1 n-heptane:EtOAc as mobile phase.

Step (b): 1-(4-Methoxy-phenyl)-1H-indole-3-carbonitrile was dissolved in dry THF and cooled to −78 ° C., then was 1.1 eq t-BuLi added drop wise and mixture stirred for one hour. A solution of 1.3 eq 1,2-dibromotetrachloroethane in dry THF was added and the mixture stirred for 4 hours while slowly warming up to rt and quenched with the addition of H₂O. The reaction mixture was diluted with DCM, phases separated and the organic phase evaporated in vacuo. The crude product was purified on silica column using 1:1 n-heptane:DCM as mobile phase.

Step (c): 2-Bromo-1-(4-methoxy-phenyl)-1H-indole-3-carbonitrile was dissolved in dry DCM and cooled to 0° C. 5 eq BBr₃, as 1.0 M solution in hexane, was added and mixture stirred over night. Still at 0° C., the reaction was quenched with the addition of MeOH. The mixture was diluted with H₂O and the phases were partitioned. The organic phase was concentrated and the crude product purified on silica column using 4:1 n-heptane:EtOAc as mobile phase.

Step (d): 2-Bromo-1-(4-hydroxy-phenyl)-1H-indole-3-carbonitrile, 1.4 eq 1H-pyrrole, 2 eq Cesium carbonate and 20 mol % Copper(I)iodide was mixed in a oven-dried vial, DMF was added and mixture flushed with nitrogen. The vial was sealed and stirred at 120° C. for 48 hours. The reaction-mixture was cooled to it, diluted with EtOAc and filtered through silica. The crude mixture was evaporated to dryness and subjected to reversed phase preparative HPLC. Appropriate fractions were combined and evaporated, and identified by 1H-NMR and LC/MS. Purity was determined by analytical HPLC.

Step (e): To a stirred solution of 1-(4-Hydroxy-phenyl)-2-pyrrol-1-yl-1H-indole-3-carbonitrile in dry DCM, under nitrogen athmosphere, at −78° C. was added DIBAL-H (3 eq, 1 M in hexane) dropwise over ca 2 minutes. The reaction mixture was stirred at ambient temperature for 2.5 hours then quenched by addition of water, acidified with 1M aq. HCl, and phases partitioned. The organic phase was evaporated to dryness and subjected to flash chromatography using 1% MeOH in DCM as mobile phase.

Step (f): The compound 1-(4-Hydroxy-phenyl)-2-pyrrol-1-yl-1H-indole-3-carbaldehyde was mixed with hydroxylamine hydrochloride (20 eq) and pyridine (20 eq) in ethanol (95%) and heated at 150° C. for 10 minutes in microwave reactor. The reaction mixture was subjected to reversed phase preparative HPLC. Appropriate fractions were combined and evaporated, and the title compound 1-(4-hydroxy-phenyl)-2-pyrrol-1-yl-1H-indole-3-carbaldehyde oxime was isolated and identified by ¹H-NMR (which showed that the product was a single isomer, but did not confirm whether the (E) or (Z) isomer had been obtained) and LC/MS. Purity was determined by analytical HPLC. ES/MS m/z: 317.9 (M+H), 316.3 (M−H); ¹H NMR (acetone-d6, 500 MHz): 8.20 (m, 1H), 7.97 (s, 1H), 7.30-7.23 (m, 2H), 7.20-7.15 (m, 3H), 6.91 (m, 2H), 6.88 (t, 2H, J=2.1 Hz) and 6.18 (t, 2H, J=2.1 Hz).

### EXAMPLE 2

### 2-(3,5-Dimethyl-isoxazol-4-yl)-1-(4-hydroxy-phenyl)-1H-indole-3-carbaldehyde oxime (E2)

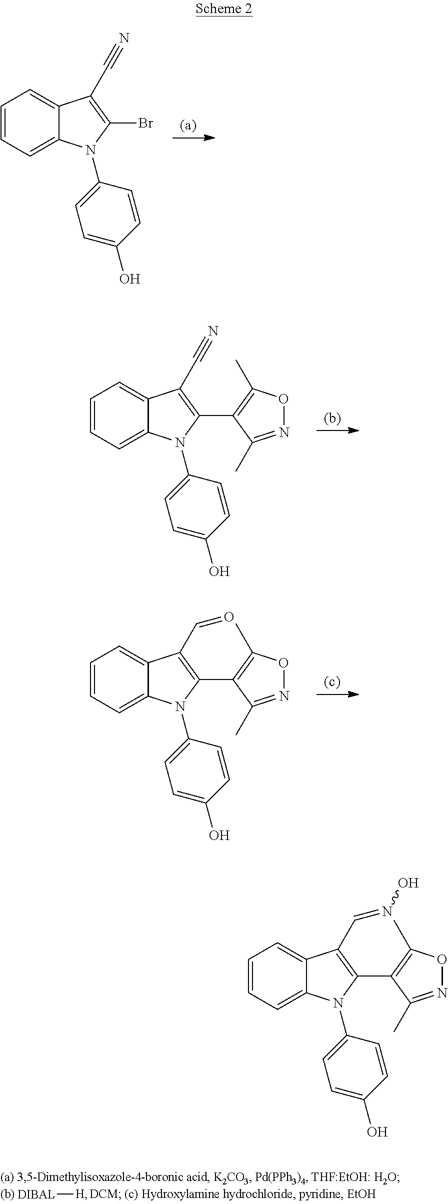

Step (a): To 2-Bromo-1-(4-hydroxy-phenyl)-1H-indole-3-carbonitrile (the intermediate product of step (c) from the synthesis of Example 1) was added 2 eq 3,5-dimethylisoxazole-4-boronic acid, 2.1 eq Potassium carbonate and 10 mol % Tetrakis(triphenylphosphine)palladium. THF:EtOH:H₂O (4:1:0.5) was added and the vial was flushed with nitrogen, sealed and stirred at 100° C. for 48 hours. The reaction mixture was cooled to rt, diluted with H₂O and extracted with EtOAc and filtered through silica. The organic phase was evaporated to dryness and subjected to reversed phase preparative HPLC. Appropriate fractions were combined and evaporated, and identified by ¹H-NMR and LC/MS. Purity was determined by analytical HPLC.

Step (b): To a stirred solution of 1-(4-Hydroxy-phenyl)-2-pyrrol-1-yl-1H-indole-3-carbonitrile in dry DCM, under nitrogen athmosphere, at −78° C. was added DIBAL-H (3 eq, 1 M in hexane) dropwise over ca 2 minutes. The reaction mixture was stirred at ambient temperature for 2.5 hours then quenched by addition of water, acidified with 1M aq. HCl, and phases were partitioned. The organic phase was evaporated to dryness and subjected to flash chromatography using 1% MeOH in DCM as mobile phase.

Step (c): 1-(4-Hydroxy-phenyl)-2-pyrrol-1-yl-1H-indole-3-carbaldehyde was mixed with hydroxylamine hydrochloride (20 eq) and pyridine (20 eq) in ethanol (95%) and heated at 150° C. for 10 minutes in microwave reactor. The reaction mixture was subjected to reversed phase preparative HPLC. Appropriate fractions were combined and evaporated, and the title compound was isolated and identified by ¹H-NMR (which showed that the product was a single isomer, but did not confirm whether the (E) or (Z) isomer had been obtained) and LC/MS. Purity was determined by analytical HPLC. ES/MS m/z: 348.19 (M+H), 346.2 (M−H); ¹H NMR (acetone-d6, 500 MHz): 8.23 (m, 1H), 7.98 (s, 1H), 7.28-7.20 (m, 3H), 7.17 (m, 2H), 6.95 (m, 2H), 2.27 (s, 3H) and 1.96 (s, 3H).

### EXAMPLE 3

### 1-(4-Hydroxy-phenyl)-2-(1H-pyrazol-3-yl)-1H-indole-3-carbaldehyde oxime (E3)

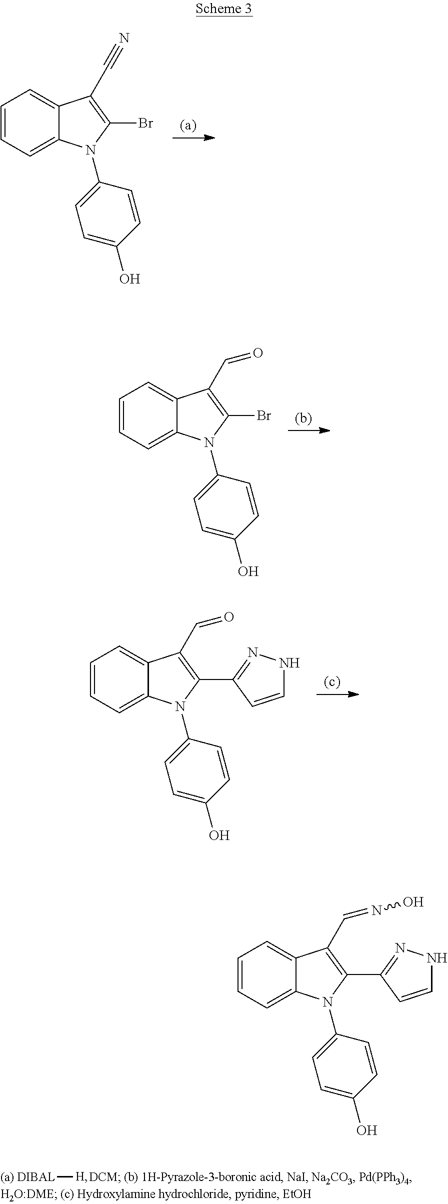

Step (a): To a stirred solution of 2-Bromo-1-(4-hydroxy-phenyl)-1H-indole-3-carbonitrile (the intermediate product of step (c) from the synthesis of Example 1, 1 g, 3.19 mmol) in CH₂Cl₂ (dry, 30 ml) at −78° C. was added diisobutylaluminium hydride (25% in hexane, 16 ml) drop wise over 10 min. The mixture was allowed to reach RT overnight and then it was stored in the freezer for 24 h. Water was added to the cooled mixture followed by HCl (6M) until acidic pH. Volatiles were removed in vacuo and the residue dissolved in CH₃OH. Purification by flash chromatography [silica, gradient, CH₂Cl₂ 100% to CH₂Cl₂:CH₃OH (95:5)] afforded 585 mg (58%) of the desired 2-Bromo-1-(4-hydroxy-phenyl)-1H-indole-3-carbaldehyde.

Step (b):To 2-bromo-1-(4-hydroxy-phenyl)-1H-indole-3-carbaldehyde (30 mg, 0.09 mmol) in a microwave vial was added 1.5 eq (0.14 mmol) 1H-Pyrazole-3-boronic acid 2 eq (28 mg, 0.19 mmol) sodium iodide, 4 eq. (40 mg, 0.38 mmol) sodium carbonate and 10 mol % (11 mg, 0.01 mmol) tetrakis(triphenylphosphine)palladium. DME (1 ml) and H₂O (0.4 ml) were added and the reaction mixture was degassed with nitrogen and irradiated in microwave reactor at 150° C. for 2.3 h . The reaction mixture was cooled to room temperature, diluted with water, extracted with ethyl acetate and concentrated to dryness. The residue was dissolved in acetonitrile and purified by reversed phase preparative HPLC. Appropriate fractions were combined and evaporated, and identified by ¹H-NMR and LC/MS. Purity was determined by analytical HPLC.

Step (c): 1-(4-Hydroxy-phenyl)-2-(1H-pyrazol-3-yl)-1H-indole-3-carbaldehyde was mixed with hydroxylamine hydrochloride (20 eq) and pyridine (20 eq) in ethanol (95%) and heated at 150° C. for 10 minutes in microwave reactor. The reaction mixture was subjected to reversed phase preparative HPLC. Appropriate fractions were combined and evaporated, and the title compound was identified by LC/MS and ¹H-NMR which showed that the product was an approximately 1:1 mixture of the (E) and (Z) isomers. Purity was determined by analytical HPLC. ES/MS m/z: 319.21 (M+H), 317.25 (M−H); ¹H NMR (acetone-d6, 500 MHz): 8.78 (br s, 1H), 8.27 (m, 1H), 7.66 (d, 1H, J=2.0 Hz), 7.22-7.16 (m, 4H), 7.09 (m, 1H), 6.98 (m, 2H) and 5.88 (br s, 1H).

### EXAMPLE 4

### 1-(4-Hydroxy-phenyl)-2-(3-methyl-3H-imidazol-4-yl)-1H-indole-3-carbaldehyde oxime (E4)

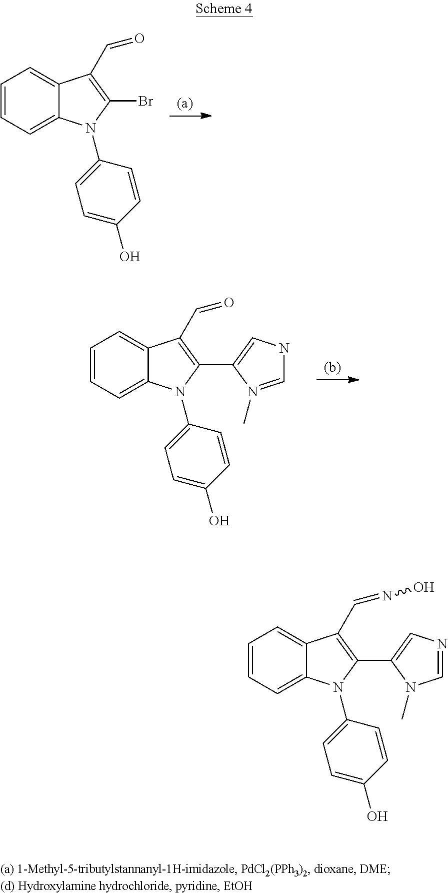

Step (a): To 2-bromo-1-(4-hydroxy-phenyl)-1H-indole-3-carbaldehyde (the intermediate product of step (a) from the synthesis of Example 3, 30 mg, 0.09 mmol) in a microwave vial was added 1.3 eq (0.12 mmol) of 1-Methyl-5-tributylstannanyl-1H-imidazole and 10 mol % (7 mg, 0.01 mmol) bis(triphenylphosphine)-palladium(II)dichloride. Dioxane (0.5 ml) and DME (0.5 ml) were added and the reaction mixture was degassed with nitrogen and irradiated in microwave reactor at 150° C. for 20 minutes. The reaction mixture was cooled to room temperature, diluted with saturated aqueous ammonium chloride solution and extracted three times with dichloromethane. The dichloromethane phase was concentrated to dryness and chromatographed by flash chromatography on silica gel using heptane:ethyl acetate gradient. Appropriate fractions were combined and evaporated to dryness. The residue was dissolved in acetonitrile and purified by reversed phase preparative HPLC. Appropriate fractions were combined and evaporated, and identified by 1H-NMR and LC/MS. Purity was determined by analytical HPLC.

Step (c): 1-(4-Hydroxy-phenyl)-2-(3-methyl-3H-imidazol-4-yl)-1H-indole-3-carbaldehyde was mixed with hydroxylamine hydrochloride (20 eq) and pyridine (20 eq) in ethanol (95%) and heated at 150° C. for 10 minutes in microwave reactor. The reaction mixture was subjected to reversed phase preparative HPLC. Appropriate fractions were combined and evaporated, and the title compound was isolated and identified by ¹H-NMR (which showed that the product was a single isomer, but did not confirm whether the (E) or (Z) isomer had been obtained) and LC/MS. Purity was determined by analytical HPLC. ES/MS m/z: 333.5 (M+H), 331.3 (M−H); ¹H NMR (acetone-d6, 500 MHz): 8.25 (m, 1H), 8.04 (s, 1H), 7.59 (s, 1H), 7.30-7.18 (m, 5H), 7.12 (d, 1H, J=1.1Hz), 6.92 (m, 2H) and 3.35 (s, 3H).

### EXAMPLES 5-8

The following compounds were prepared according to General Method I above. Full experimental details of the individual steps of that general method are described in Examples 1 and 2 above. For each of Examples 5-8, the title compound was identified by ‘H-NMR which showed that the product was a single isomer, but did not confirm whether the (E) or (Z) isomer had been obtained.

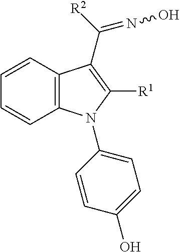

| | |
|---|---|
| E 5 | 1-(4-Hydroxy-phenyl)-2-phenyl-1H-indole-3-carbaldehyde oxime |
| R¹ = Phenyl | R² = H |

ES/MS m/z: 329.18 (pos. M + H), 327.21 (neg. M − H); ¹H NMR (methanol-d4, 500 MHz): 8.23 (m,
1H), 8.11 (s, 1H), 7.36-7.33 (m, 3H), 7.29-7.26 (m, 2H), 7.23-7.17 (m, 2H), 7.14 (m, 1H), 7.03 (m, 2H)
and 6.79 (m, 2H).

| | |
|---|---|
| E 6 | 1-(4-Hydroxy-phenyl)-1H-indole-3-carbonitrile |
| R¹ = Thiophene-2-yl | R² = H |

ES/MS m/z: 335.13 (pos. M + H), 333.16 (neg. M − H); ¹H NMR (acetone-d6, 500 MHz): 8.33 (s, 1H),
8.25 (m, 1H), 7.60 (dd, 1H, J = 4.7, 1.6 Hz), 7.26-7.20 (m, 2H), 7.18 (m, 2H), 7.13-7.09 (m, 3H) and 6.95
(m, 2H).

| | |
|---|---|
| E 7 | 1-(4-Hydroxy-phenyl)-2-thiophen-3-yl-1H-indole-3-carbaldehyde oxime |
| R¹ = Thiophen-3-yl | R² = H |

ES/MS m/z: 335.12 (pos. M + H), 333.17 (neg. M − H); ¹H NMR (acetone-d6, 500 MHz): 8.25 (s, 1H),
8.24 (m, 1H), 7.50-7-47 (m, 2H), 7.24-7.18 (m, 2H), 7.15 (m, 2H), 7.12 (m, 1H) and 6.95-6.92 (m, 3H).

| | |
|---|---|
| E 8 | 1-(4-Hydroxy-phenyl)-2-(3-methyl-thiophen-2-yl)-1H-indole-3-carbaldehyde oxime |
| R¹ = 3-Methyl-thiophen-2-yl | R² = H |

ES/MS m/z: 349.12 (pos. M + H), 347.16 (neg. M − H); ¹H NMR (acetone-d6, 500 MHz): 8.24 (m, 1H),
8.05 (s, 1H), 7.51 (d, 1H, J = 5.1 Hz), 7.27-7.17 (m, 3H), 7.14 (m, 2H), 6.93 (d, 1H, J = 5.1 Hz), 6.90 (m,
2H) and 2.07 (s, 3H).

### EXAMPLE 9

### 1-[1-(4-Hydroxy-phenyl)-2-phenyl-1H-indol-3-yl]ethanone oxime (E9)

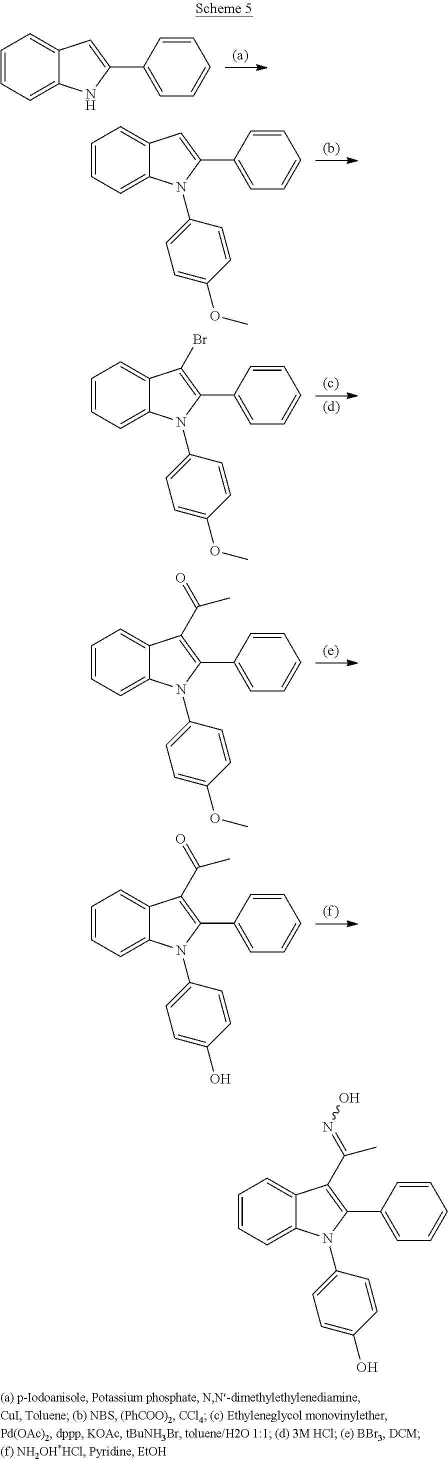

Step (a): A flask with a mixture of 2-phenylindole (2.90 g, 15.0 mmol, 1 eq.), 4-iodoanisole (4.21 g, 18.0 mmol, 1.2 eq.), cupruous iodide (144 mg, 0.76 mmol, 0.05 eq.), and potassium phosphate, tribasic (6.69 g, 31.5 mmol, 2 eq.) was evacuated and refilled with argon three times. To this degassed mixture of solids was added 15 mL of dry toluene under argon. Argon was bubbled through this suspension, and N,N′-ethylenediamine (0.32 mL, 3.0 mmol, 0.2 eq.) added as neat liquid under argon. The flask was closed with a reflux condenser and an argon inlet; the green suspension was heated to 110° C. whereupon it turned red. The red suspension was heated overnight at 110° C. with stirring. The reaction mixture was then cooled to room temperature, and 30 mL of ethyl acetate was added. This mixture was filtered through a short silica column; the column was rinsed with ethyl acetate until no more product came out by TLC, and the solvents evaporated under reduced pressure. The crude product was recrystallized from ethyl acetate. The mother liquor was concentrated, and the residual substance recrystallized from ethyl acetate. This way three crops were collected. They were mixed and homogenized. Yield of 1-(4-methoxyphenyl)-2-phenylindole 3.96 g (88%) as light orange crystalline substance. This was used directly in the next step.

Step (b): To a stirred suspension of 1-(4-methoxyphenyl)-2-phenyl-1H-indole (3.82 g, 12.8 mmol, 1 eq.) in 15 mL of CCl₄ was added N-bromosuccinimide (2.28 g, 12.8 mmol, 1 eq.) and the resulting red suspension was stirred at 0° C. Then about 50 mg of benzoyl peroxide was added, and stirring at 0° C. continued for 20 minutes. The dark red suspension was allowed to warm to room temperature with stirring. After 15 minutes at room temperature, the red suspension was filtered, the N-succinimide precipitate washed with CCl₄ on the filter, and the combined filtrates were evaporated. The crude 3-bromo-1-(4-methoxyphenyl)-2-phenylindole was purified by silica column eluted with 9:1 to 6:1 petroleum ether/ethyl acetate. The substance from the column was recrystallized from ethyl acetate/petroleum ether to give 3-bromo-1-(4-methoxyphenyl)-2-phenyl-1H-indole (3.13 g, 65%) as pink crystals. Further recrystallization gave yellowish crystals. Yield 55% over 2 steps.

Step (c) and (d): 3-bromo-1-(4-methoxyphenyl)-2-phenyl-1H-indole (100 mg, 0.26 mmol), 2-(vinyloxy)ethanol (116 mg, 1.32 mmol), palladium acetate (3 mg, 0.01 mmol), K₂CO₃(48 mg, 0.34 mmol) and tetrabutyl ammonium bromide (4 mg, 0.01 mmol) were mixed with toluene (0.5 ml) and water (0.5 ml) in a microwave vial under nitrogen. The reaction was run at 150° C. for 20 min. 2 ml 3M HCl was added and the mixture was stirred at RT for 30 min. Water and DCM were added and the phases were separated. After evaporation of the solvents, the residue was purified by flash chromatography with Heptane/EtOAc 9:1 as eluent, to provide 50 mg (55%) of 1-(1-(4-methoxyphenyl)-2-phenyl-1H-indol-3-yl)ethanone.

Step (e): 0.11 ml of a 1M DCM solution of BBr₃ was added to a cooled (−78° C.) DCM solution of 1-(1-(4-methoxyphenyl)-2-phenyl-1H-indol-3-yl)ethanone (12 mg, 0.04 mmol) under nitrogen. The temperature was allowed to RT and the mixture was stirred for 2 h. The reaction was quenched by water and the phases were separated. After evaporation of the solvents, the residue was purified by prep-HPLC to provide 6.5 mg (56%) of 1-(1-(4-hydroxyphenyl)-2-phenyl-1H-indol-3-yl)ethanone.

Step (f): 1-(1-(4-hydroxyphenyl)-2-phenyl-1H-indol-3-yl)ethanone (10 mg, 0.03 mmol), hydroxylamine hydrochloride (42 mg, 0.61 mmol) and pyridine (20 μl) were mixed in 99.7% EtOH. The reaction was run at 150° C. for 10 min. The product was purified by prep-HPLC to provide 1 mg (10%) of the title compound 1-(1-(4-hydroxyphenyl)-2-phenyl-1H-indol-3-yl)ethanone oxime. Identification by ¹H-NMR showed that the product was a single isomer, but did not confirm whether the (E) or (Z) isomer had been obtained. ES/MS m/z: 343.18 (pos. M+H), 341.19 (neg. M−H); ¹H NMR (acetone-d6, 500 MHz): 7.54 (m, 1H), 7.33-7.22 (m, 5H), 7.17-7.14 (m, 2H), 7.12 (m, 1H), 7.08 (m, 2H), 6.88 (m, 2H), and 2.10 (s, 3H).

### EXAMPLES 10-18

The following compounds were prepared according to General Method I above. Full experimental details of the individual steps of that general method are described in Examples 1 and 2 above. For each of Examples 10-18, the title compound was identified by ‘H-NMR which showed that the product was a single isomer, but did not confirm whether the (E) or (Z) isomer had been obtained.

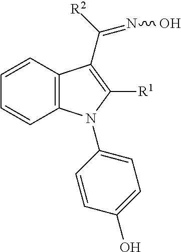

| | |
|---|---|
| E 10 | 1-(4-Hydroxy-phenyl)-2-(4-methyl-thiophen-3-yl)-1H-indole-3-carbaldehyde oxime |
| R¹ = 4-Methyl-thiophen-3-yl | R² = H |

ES/MS m/z: 349.16 (pos. M + H), 347.24 (neg. M − H); ¹H NMR (acetone-d6, 500 MHz): 8.23 (m, 1H),
7.97 (s, 1H9, 7.52 (d, 1H, J = 3.2 Hz), 7.25-7.19 (m, 3H), 7.15 (m, 2H), 7.12 (m, 1H), 6.89 (m, 2H) and
1.92 (d, 3H; J = 0.9 Hz).

| | |
|---|---|
| E 11 | 2-(3,5-Dimethyl-1H-pyrazol-4-yl)-1-(4-hydroxy-phenyl)-1H-indole-3-carbaldehyde oxime |
| R¹ = 3,5-Dimethyl-1H-pyrazol-4-yl | R² = H |

ES/MS m/z: 347.2 (pos. M + H), 345.29 (neg. M − H); ¹H NMR (acetone-d6, 500 MHz): 8.22 (m, 1H),
7.95 (s, 1H), 7.21-7.16 (m, 3H), 7.10 (m, 2H), 6.90 (m, 2H) and 2.00 (s, 6H).

| | |
|---|---|
| E 12 | 1-(4-Hydroxy-phenyl)-2-(5-methyl-1H-pyrazol-4-yl)-1H-indole-3-carbaldehyde oxime |
| R¹ = 5-Methyl-1H-pyrazol-4-yl | R² = H |

ES/MS m/z: 333.21 (pos. M + H), 331.3 (neg. M − H); ¹H NMR (acetone-d6, 500 MHz): 8.20 (m, 1H),
8.02 (s, 1H), 7.47 (br s, 1H), 7.20-7.09 (m, 5H), 6.86 (m, 2H) and 2.02 (s, 3H).

| | |
|---|---|
| E 13 | 1-(4-Hydroxy-phenyl)-2-(2-methyl-2H-pyrazol-3-yl)-1H-indole-3-carbaldehyde oxime |
| R¹ = 2-Methyl-2H-pyrazol-3-yl | R² = H |

ES/MS m/z: 333.5 (pos. M + H), 331.3 (neg. M − H); ¹H NMR (acetone-d6, 500 MHz): (acetone-d6,
500 MHz): 8.25 (m, 1H), 8.00 (s, 1H), 7.45 (d, 1H, J = 1.9 Hz), 7.31-7.23 (m, 3H), 7.19 (m, 2H), 6.92 (m,
2H), 6.43 (d, 1H; J = 1.9 Hz) and 3.55 (s, 3H).

| | |
|---|---|
| E 14 | 2-[3-(Hydroxyimino-methyl)-1-(4-hydroxy-phenyl)-1H-indol-2-yl]-furan-3-carbonitrile |
| R¹ = 3-Cyano-furan-2-yl | R² = H |

ES/MS m/z: 344 (pos. M + H); ¹H NMR (acetone-d6, 500 MHz): 8.24 (m, 1 H), 8.09 (s, 1H), 7.58 (d,
1H, J = 2.1 Hz), 7.28 (m, 1H), 7.24-7.20 (m, 4H), 6.89 (m, 2H) and 6.73 (d, 1H, J = 2.1 Hz).

| | |
|---|---|
| E 15 | 2-[3-(Hydroxyimino-methyl)-1-(4-hydroxy-phenyl)-1H-indol-2-yl]thiophene-3-carbonitrile |
| R¹ = 3-Cyano-thiophene-2-yl | R² = H |

ES/MS m/z: 360.2 (pos. M + H); ¹H NMR (acetone-d6, 500 MHz): 8.21 (m, 1H), 8.11 (s, 1H), 7.58 (d,
1H, J = 5.3 Hz), 7.32 (d, 1H, J = 5.3 Hz), 7.26-7.18 (m, 4H), 7.13 (m, 1H) and 6.85 (m, 2H).

| | |
|---|---|
| E 16 | 5-[3-(Hydroxyimino-methyl)-1-(4-hydroxy-phenyl)-1H-indol-2-yl]-1-methyl-1H-pyrazole-4- |
| | carbonitrile |
| R¹ = 4-Cyano-1-methyl-pyrazole-5-yl | R² = H |

ES/MS m/z: 358.4 (pos. M + H), 356.2 (neg. M − H); ¹H NMR (acetone-d6, 500 MHz): 8.27 (m, 1H),
8.10 (s, 1H), 7.98 (s, 1H), 7.36 (m, 1H), 7.32-7.27 (m, 2H), 7.22 (m, 2H), 6.97 (m, 2H) and 3.72 (s, 3H).

| | |
|---|---|
| E 17 | 2-[3-(Hydroxyimino-methyl)-1-(4-hydroxy-phenyl)-1H-indol-2-yl]-benzonitrile |
| R¹ = 2-cyano-phenyl | R² = H |

ES/MS m/z: 354.2 (pos. M + H), 352.4 (neg. M − H); ¹H NMR (acetone-d6, 500 MHz): 8.27 (m, 1H),
7.98 (s, 1H), 7.83 (m, 1H), 7.76 (m, 1H), 7.67 (m, 1H), 7.63 (m, 1H), 7.31-7.16 (m, 5H) and 6.88 (m,

| | |
|---|---|
| 2H). | |
| E 18 | 1-Ethyl-2-[3-(hydroxyimino-methyl)-1-(4-hydroxy-phenyl)-1H-indol-2-yl]-1H-pyrrole-3- |
| | carbonitrile |
| R¹ = 4-Cyano-1-ethyl-pyrazole-5-yl | R² = H |

ES/MS m/z: 371.3 (pos. M + H), 369.1 (neg. M − H); ¹H NMR (acetone-d6, 500 MHz): 8.28 (m, 1H),
8.00 (s, 1H), 7.32 (m, 1H), 7.28-7.18 (m, 4H), 7.05 (d, 1H; J = 3.1 Hz), 6.95 (m, 2H), 6.58 (d, 1H,
J = 3.1 Hz), 3.79 (m, 1H), 3.70 (m, 1H) and 1.14 (t, 3H; J = 7.1 Hz).

### EXAMPLES 19-25

The following compounds were prepared according to General Method III above. Full experimental details of the individual steps of that general method are described in Example 31 below (if not stated otherwise R³, R⁴, R⁵ and R⁶ are hydrogen). For each of Examples 20-24, the title compound was identified by ¹H-NMR which showed that the product was a single isomer, but did not confirm whether the (E) or (Z) isomer had been obtained. For Example 25, the title compound was identified by ¹H-NMR which showed that the product was an approximately 1:1 mixture of the (E) and (Z) isomers. For Example 19, the ¹H-NMR was unclear as to whether the the product was a single isomer, or whether it was a mixture of the (E) and (Z) isomers.

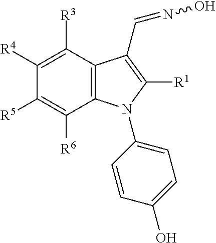

| | |
|---|---|
| E 19 | 4-Fluoro-1-(4-hydroxy-phenyl)-2-pyrrol-1-yl-1H-indole-3-carbaldehyde oxime |
| R¹ = Pyrrol-1-yl | R³ = Fluoro |

ES/MS m/z: 336.17 (pos. M + H), 334.18 (neg. M − H); ¹H NMR (acetone-d6, 500 MHz): 8.15 (s, 1H),
7.24 (m, 1H), 7.19 (m, 2H), 6.97-6.93 (m, 2H), 6.90 (m, 2H), 6.84 (t, 2H J = 2.2 Hz) and 6.11 (t, 2H,
J = 2.2 Hz).

| | |
|---|---|
| E 20 | 4-Fluoro-1-(4-hydroxy-phenyl)-2-phenyl-1H-indole-3-carbaldehyde oxime |
| R¹ = Phenyl | R³ = Fluoro |

ES/MS m/z: 347.15 (pos. M + H), 345.18 (neg. M − H); ¹H NMR (acetone-d6, 500 MHz): 8.15 (s, 1H),
7.24 (m, 1H), 7.19 (m, 2H), 6.97-6.93 (m, 2H), 6.90 (m, 2H), 6.84 (t, 2H J = 2.2 Hz) and 6.11 (t, 2H,
J = 2.2 Hz).

| | |
|---|---|
| E 21 | 2-(3,5-Dimethyl-isoxazol-4-yl)-7-fluoro-1-(4-hydroxy-phenyl)-1H-indole-3-carbaldehyde |
| | oxime |
| R¹ = 3,5-Dimethyl-isoxazol-4-yl | R⁶ = Fluoro |

ES/MS m/z: 366.17 (pos. M + H), 364.15 (neg. M − H); ¹H NMR (Methanol-d4, 500 MHz): 8.03-8.18
(d, 1H, J = 8 Hz); 7.9 (s, 1H); 7.17-7.10 (m, 2H); 7.04 (m, 1H); 6.98-6.94 (m, 1H); 6.79 (m, 2H); 2.22 (s,
3H); 1.97 (s, 3H)

| | |
|---|---|
| E 22 | 2-(3,5-Dimethyl-isoxazol-4-yl)-5-fluoro-1-(4-hydroxy-phenyl)-1H-indole-3-carbaldehyde |
| | oxime |
| R¹ = 3,5-Dimethyl-isoxazol-4-yl | R⁴ = Fluoro |

ES/MS m/z: 366.18 (pos. M + H), 364.25 (neg. M − H); ¹H NMR (acetone-d6, 500 MHz): 7.96 (s, 1H),
7.89 (dd, 1H, J = 9.9, 2.8 Hz), 7.23-7.15 (m, 3H), 7.07 (m, 1H), 6.96 (m, 2H), 2.78 (s, 3H) and 1.96 (s,
3H).

| | |
|---|---|
| E 23 | 5-Fluoro-1-(4-hydroxy-phenyl)-2-phenyl-1H-indole-3-carbaldehyde oxime |
| R¹ = Phenyl | R⁴ = Fluoro |

ES/MS m/z: 347.17 (neg. M − H), 345.28 (neg. M − H); ¹H NMR (acetone-d6, 500 MHz): 8.12 (s, 1H),
7.92 (dd, 1H, J = 9.9, 2.6 Hz), 7.42-7.38 (m, 3H), 7.34 (m, 2H), 7.16-7.12 (m, 3H), 7.04 (m, 1H) and 6.89
(m, 2H).

| | |
|---|---|
| E 24 | 5-Fluoro-1-(4-hydroxy-phenyl)-2-pyrrol-1-yl-1H-indole-3-carbaldehyde oxime |
| R¹ = Pyrrol-1-yl | R⁴ = Fluoro |

ES/MS m/z: 336.18 (pos. M + H), 334.24 (neg. M − H); ¹H NMR (acetone-d6, 500 MHz): 7.96 (s, 1H),
7.86 (dd, 1H, J = 9.6, 2.7 Hz), 7.21-7.16 (m, 3H), 7.09 (m, 1H), 6.91 (m, 2H), 6.88 (t, 2H, J = 2.2 Hz) and
6.19 (t, 2H, J = 2.2 Hz).

| | |
|---|---|
| E 25 | 2-(3,5-Dimethyl-isoxazol-4-yl)-4-fluoro-1-(4-hydroxy-phenyl)-1H-indole-3-carbaldehyde |
| | oxime |
| R¹ = 3,5-Dimethyl-isoxazol-4-yl | R³ = Fluoro |

ES/MS m/z: 366.2 (pos. M + H), 364.27 (neg. M − H); ¹H NMR (acetone-d6, 500 MHz): 8.47 (d, 1H,
J = 1.2 Hz), 7.23-7.16 (m, 3H), 7.01 (d, 1H, J = 8.5 Hz), 6.97-6.92 (m, 3H), 2.17 (s, 3H) and 1.98 (s, 3H).

### EXAMPLES 26-30

The following compounds were prepared according to General Method I above. Full experimental details of the individual steps of that general method are described in Examples 1 and 2 above (if not stated otherwise R⁷, R⁸, R⁹ and R¹⁰ are hydrogen). For each of Examples 26, 29 and 30, the title compound was identified by ¹H-NMR which showed that the product was a single isomer, but did not confirm whether the (E) or (Z) isomer had been obtained. For Examples 27 and 28, the title compound was identified by ¹H-NMR which showed that the product was an approximately 1:1 mixture of the (E) and (Z) isomers.

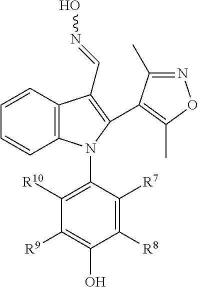

| | |
|---|---|
| E 26 | 1-(3-Chloro-4-hydroxy-phenyl)-2-(3,5-dimethyl-isoxazol-4-yl)-1H-indole-3-carbaldehyde |
| | oxime |
| R⁸ = Chloro | |

ES/MS m/z: 382.17 (pos. M + H), 381.32 (neg. M − H); ¹H NMR (acetone-d6, 500 MHz): 8.23 (m, 1H),
7.98 (s, 1H), 7.37 (br s, 1H), 7.30-7.22 (m, 3H), 7.13 (br s, 2H), 2.31 (s, 3H) and 2.00 (s, 3H).

| | |
|---|---|
| E 27 | 2-(3,5-Dimethyl-isoxazol-4-yl)-1-(2-fluoro-4-hydroxy-phenyl)-1H-indole-3-carbaldehyde |
| | oxime |
| R⁷ = Fluoro | |

ES/MS m/z: 366.17 (pos. M + H), 364.24 (neg. M − H); ¹H NMR (acetone-d6, 500 MHz): Two
conformations, some peaks are split 8.23 (m, 1H), 7.99 and 7.98 (split, s, 1H), 7.38-7.23 (m, 3H), 7.11
(m, 1H), 6.85-6.75 (m, 2H), 2.26 and 2.24 (split, s, 3H) and 2.02 and 2.00 (split, s 3H).

| | |
|---|---|
| E 28 | 1-(2,5-Difluoro-4-hydroxy-phenyl)-2-(3,5-dimethyl-isoxazol-4-yl)-1H-indole-3-carbaldehyde |
| | oxime |
| R⁷ = Fluoro | R⁹ = Fluoro |

ES/MS m/z: 320.3 (pos. M + H), 318.1 (neg. M − H); ¹H NMR (acetone-d6, 500 MHz): 7.43 (m, 1H),
7.31 (m, 2H), 7.17 (m, 1H), 7.09-7.06 (m, 3H), 6.93 (m, 1H), 3.15 (m, 2H), 3.00 (s, 3H), 1.45 (m, 2H),
1.20 (m, 2H) and 0.83 (t, 3H, J = 7.4 Hz).

| | |
|---|---|
| E 29 | 2-(3,5-Dimethyl-isoxazol-4-yl)-1-(3-fluoro-4-hydroxy-phenyl)-1H-indole-3-carbaldehyde |
| | oxime |
| R⁸ = Fluoro | |

ES/MS m/z: 366.21 (pos. M + H), 364.25 (neg. M − H); ¹H NMR (acetone-d6, 500 MHz): 8.23 (m, 1H),
7.98 (s, 1H), 7.30-7.23 (m, 3H), 7.21-7.11 (m, 2H), 7.03 (m, 1H), 2.30 (s, 3H) and 1.99 (s, 3H).

| | |
|---|---|
| E 30 | 1-(3,5-Difluoro-4-hydroxy-phenyl)-2-(3,5-dimethyl-isoxazol-4-yl)-1H-indole-3-carbaldehyde |
| | oxime |
| R⁸ = Fluoro | R⁹ = Fluoro |

ES/MS m/z: 384.24 (pos. M + H), 382.24 (neg. M − H); ¹H NMR (acetone-d6, 500 MHz): 8.23 (m, 1H),
7.97 (s, 1H), 7.31-7.24 (m, 3H), 7.05 (m, 2H), 2.33 (s, 3H) and 2.02 (s, 3H).

### EXAMPLE 31

### 2-(3,5-dimethylisoxazol-4-yl)-5,6-difluoro-1-(4-hydroxyphenyl)-1H-indole-3-carbaldehyde oxime (E31)

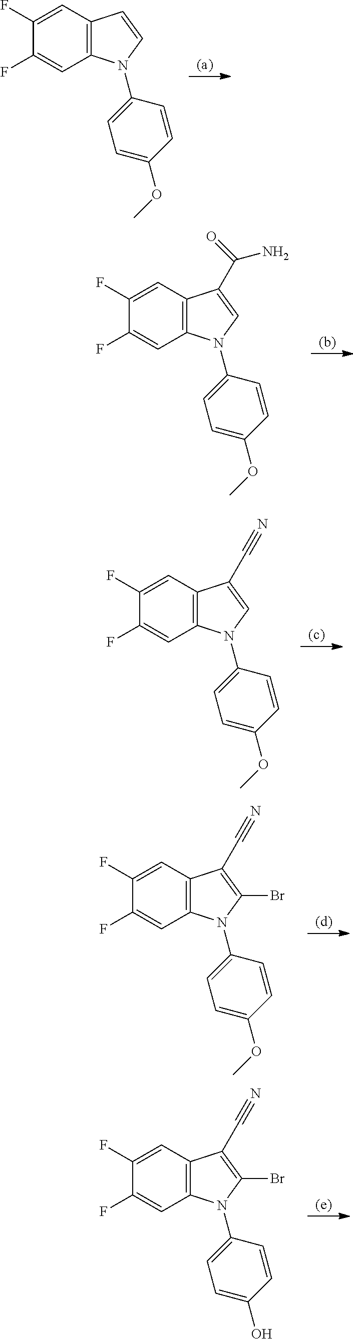

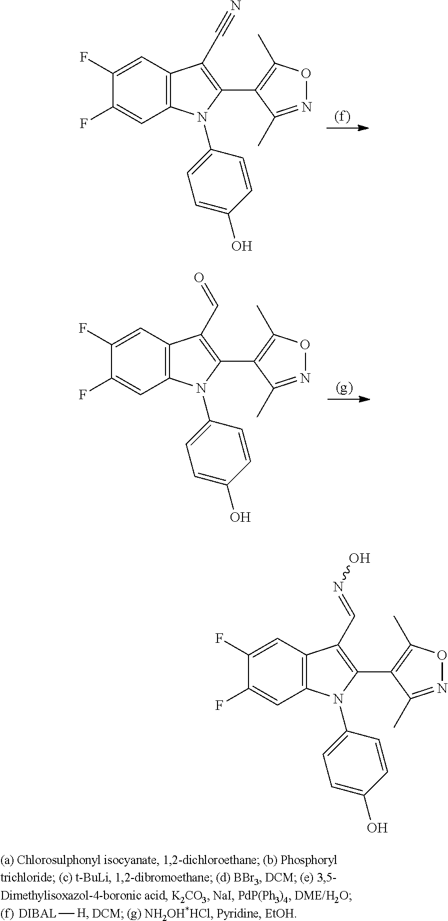

Step (a): To a stirred solution of 5,6-difluoro-1-(4-methoxyphenyl)-1H-indole (synthesized from 5,6-difluoro-1H-indole and p-iodoanisole using a procedure analogous to that described in Step (a) of Example 1) (360 mg, 1.39 mmol) in 7 ml of 1,2-dichloroethane, chlorosulphonyl isocyanate (145 μl, 1.67 mmol) was added at RT and the mixture stirred at this temperature for 2 h. Water was added, pH was adjusted to 8 with an aq. solution 2M NaOH. The mixture was extracted with EtOAc. The combined organic layers were washed with brine, dried over MgSO₄ and concentrated in vacuo. LC/MS indicated that the wanted product, 5,6-difluoro-1-(4-methoxyphenyl)-1H-indole-3-carboxamide was formed. The product was used as such in the next step without further purification.

Step (b): 5,6-difluoro-1-(4-methoxyphenyl)-1H-indole-3-carboxamide (420 mg, 1.39 mmol) was dissolved in 15 ml of phosphoryl trichloride and stirred at 65° C. for 2 h. The mixture was allowed to cool to room temperature and diluted with toluene. Concentrated in vacuo and co-evaporated once more with toluene. The residue was dissolved in DCM, washed with sat. aq. solution of NaHCO₃. The volatiles were removed in vacuuo and the residue was purified by flash chromatography [silica; gradient: n-heptane 100% to n-heptane-AcEt (6:1)]. 132 mg of the desired product, 5,6-difluoro-1-(4-methoxyphenyl)-1H-indole-3-carbonitrile was obtained (33% over 2 steps).

Step (c): 5,6-difluoro-1-(4-methoxyphenyl)-1H-indole-3-carbonitrile (132 mg, 0.46 mmol) was dissolved in 8 ml of dry THF and stirred at −78° C. To this solution, tert-butyllithium (1.7 mmol in pentane, 0.99 mmol) was added slowly. The mixture was allowed to reach −50° C. and stirred at that temperature during 30 min. 1,1′,2,2′-tetrachloro-1,2-dibromo-ethane (242 mg, 0.74 mmol) dissolved in 3 ml of dry THF was added and the mixture was stirred at −78° C. for 1 hour, and then at for RT 3 h. Sat. aq NH₄Cl was added and the volatiles removed in vacuuo. The residue was extracted with ethyl acetate, the organic layer was washed with brine and dried over MgSO₄ and concentrated in vacuuo. The residue obtained was used directly in the next step.

Step (d): 170 mg of the mixture obtained in the step before was dissolved in 8 ml of dry DCM and cooled to −78° C. To this stirred mixture, BBr₃ (1 M in DCM, 1.87 mmol) was added slowly. The reaction was left in the fridge O.N. Few drops MeOH were added followed by brine. The phases were partitioned, then concentrated in vacuo. Purification by flash chromatography [silica; n-hepatne-EtOAc (4:1)] afforded 123 mg of the wanted 2-bromo-5,6-difluoro-1-(4-hydroxyphenyl)-1H-indole-3-carbonitrile. Yield=76% over 2 steps.

Step (e): 2-bromo-6-fluoro-1-(4-hydroxyphenyl)-1H-indole-3-carbonitrile (123 mg, 0.35 mmol), 3,5-dimethylisoxazol-4-ylboronic acid (99 mg, 0.70 mmol), K₂CO₃ (243 mg, 1.76 mmol), NaI (199 mg, 0.70 mmol) and tetrakis(triphenylphosphine)Pd(0) (20 mg, 0.02 mmol) were mixed in a microwave vial, suspended in 3 ml DME/water (1:1) and heated in the microwave at 150° C. for 15 min. The mixture was filtered through a prepacked silica column and evaporated in vacuuo. The reaction was purified by flash chromatography [silica; n-heptane-EtOAc (4:1)] to afford 56 mg of the desired 2-(3,5dimethylisoxazol-4-yl)-5,6-difluoro-1-(4-hydroxyphenyl)-1H-indole-3-carbonitrile (43%).

Step (f): 2-(3,5dimethylisoxazol-4-yl)-5,6-difluoro-1-(4-hydroxyphenyl)-1H-indole-3-carbonitrile (28 mg, 0.08 mmol) was dissolved in 3 ml of dry DCM and cooled to −78° C. To this stirred solution, DIBAL-H (1M in Hexane, 0.38 mmol) was added slowly. The mixture was allowed to reach RT and stirred at that temperature for 24 h. Water was added to the mixture and then it was acidified with HCl (2 M). The mixture was evaporated in vacuuo. The residue dissolved in acetone and filtered through a silica plug. Purification by semi-preparative HPLC afforded 11 mg (39%) of the desired 2-(3,5-dimethylisoxazol-4-yl)-5,6-difluoro-1-(4-hydroxyphenyl)-1H-indole-3-carbaldehyde.

Step (g): 2-(3,5-dimethylisoxazol-4-yl)-5,6-difluoro-1-(4-hydroxyphenyl)-1H-indole-3-carbaldehyde (11 mg, 0.03 mmol), hydroxylamine hydrochloride (69.5 mg, 0.60 mmol) and 50 μl of pyridine were suspended in 2 ml of EtOH (95%). This mixture was heated at 150° C. in the microwave during 5 min. The reaction were filtered and purified by semi-preparative to afford 3 mgs (26%) of the desired 2-(3,5-dimethylisoxazol-4-yl)-5,6-difluoro-1-(4-hydroxyphenyl)-1H-indole-3-carbaldehyde oxime. Identification by ¹H-NMR showed that the product was a single isomer, but did not confirm whether the (E) or (Z) isomer had been obtained. ES/MS m/z: 384.1 (pos. M+H), 382.1 (neg. M−H); ¹H NMR (acetone-d6, 500 MHz): 8.03 (dd, 1H, J=11.2, 8.1 Hz), 7.95 (s, 1H), 7.19 (m, 2H), 7.13 (dd, 1H, J=11.2, 7.0 Hz), 6.96 (m, 2H), 2.28 (s, 3H) and 1.95 (s, 3H).

### EXAMPLES 32 AND 33

### (E)-1-(2-(3,5-dimethylisoxazol-4-yl)-1-(4-hydroxyphenyl)-1H-indol-3-yl)-2,2,2-trifluoroethanone oxime (E32) and (Z)-1-(2-(3,5-dimethylisoxazol-4-yl)-1-(4-hydroxyphenyl)-1H-indol-3-yl)-2,2,2-trifluoroethanone oxime (E33)

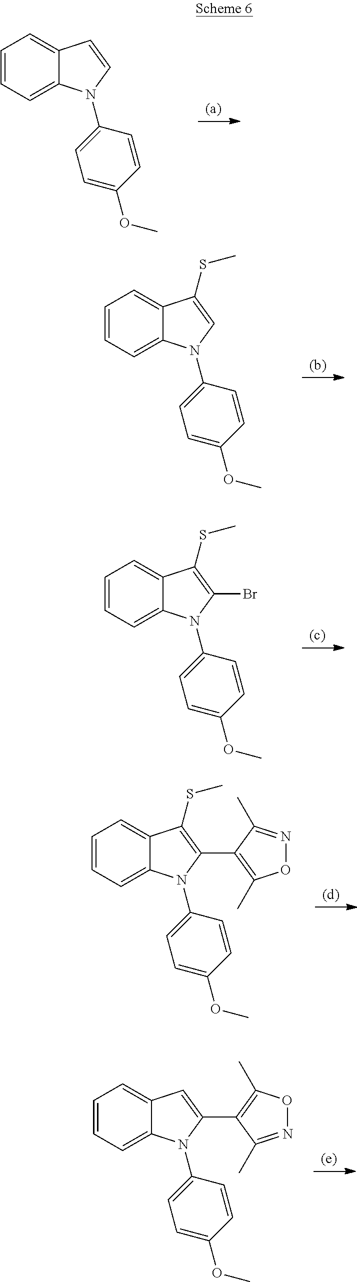

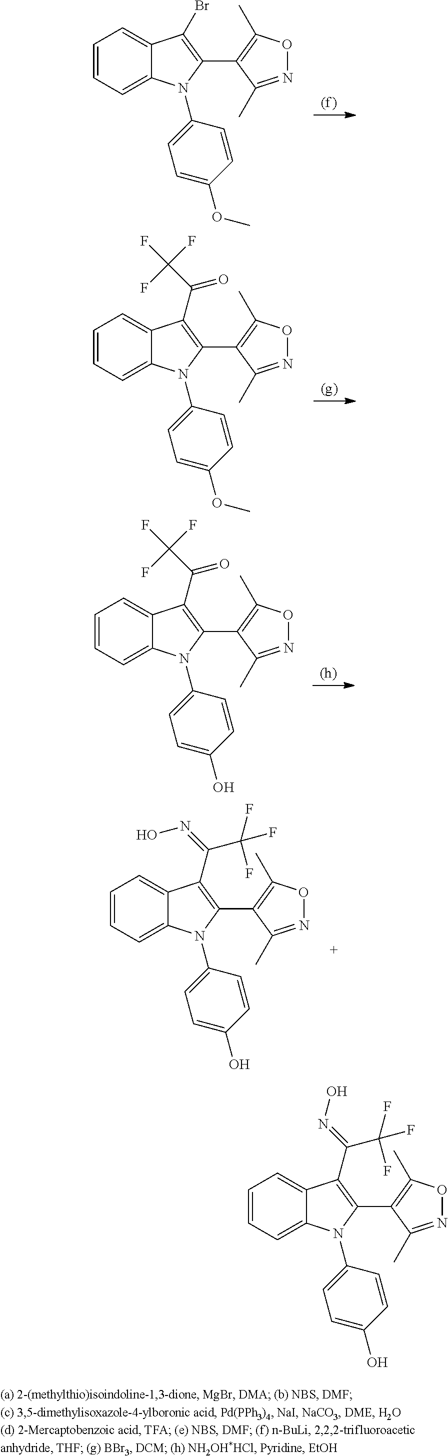

Step (a): 1-(4-methoxyphenyl)-1H-indole (prepared using the synthesis described in J. Org. Chem. 2008, 73 (14), 5529-5535) (1.0 g, 4.48 mmol), 2-(methylthio)isoindoline-1,3-dione (0.95 g, 4.93 mmol) and magnesium bromide (8 mg, 0.045 mmol) were mixed in degassed DMA and stirred under an atmosphere of nitrogen at 90° C. for 90 min. 1M NaOH and EtOAc were added. The phases were separated and the organic solvents were evaporated. The residue was purified by flash chromatography with Heptane/EtOAc 20:1 to provide 1-(4-methoxyphenyl)-3-(methylthio)-1H-indole in 80% yield. ES/MS m/z: 270.11 (M+H).

Step (b): NBS (529 mg, 2.97 mmol) was added to a cooled (0° C.) solution of 1-(4-methoxyphenyl)-3-(methylthio)-1H-indole (800 mg, 2.97 mmol) in 10 ml of DMF. The temperature was allowed to RT and the mixture was stirred at RT for 30 min. Water and DCM were added and the phases were separated. After evaporation of the solvents, the residue was purified by flash chromatography with heptane/EtOAc 20:1 to provide 2-bromo-1-(4-methoxyphenyl)-3-(methylthio)-1H-indole in 66% yield. ES/MS m/z: 348.04, 350.01 (M+H).

Step (c): 4-(1-(4-methoxyphenyl)-3-(methylthio)-1H-indol-2-yl)-3,5-dimethylisoxazole was synthesized from 2-bromo-1-(4-methoxyphenyl)-3-(methylthio)-1H-indole using a procedure analogous to that decribed in step (a) from Example 2.

Step (d): 4-(1-(4-methoxyphenyl)-3-(methylthio)-1H-indol-2-yl)-3,5-dimethylisoxazole (140 mg, 0.38 mmol) and 2-mercaptobenzoic acid (118 mg, 0.77 mmol) were added to 5 ml of trifluoroacetic acid at RT. The mixture was stirred as a slurry at RT under an atmosphere of nitrogen over night. 2M NaOH and EtOAc were added and the phases were separated. The solvents were evaporated and the residue was purified by flash chromatography with heptane/EtOAc 4:1 as eluent to provide 4-(1-(4-methoxyphenyl)-1H-indol-2-yl)-3,5-dimethylisoxazole in 86% yield. ES/MS m/z: 319.1 (M+H)

Step (e): NBS (59 mg, 0.33 mmol) was added to a cooled (0° C.) solution of 4-(1-(4-methoxyphenyl)-1H-indol-2-yl)-3,5-dimethylisoxazole (105 mg, 0.33 mmol) in 5 ml of DMF. The temperature was allowed to RT and the mixture was stirred at RT for 30 min. DMF was evaporated. DCM and water were added and the phases were separated. After evaporation of the solvents, the residue was purified by flash chromatography with heptane/EtOAc 9:1 to provide 4-(3-bromo-1-(4-methoxyphenyl)-1H-indol-2-yl)-3,5-dimethylisoxazole in 98% yield. ES/MS m/z: 365.14 (M+H), 363.30 (M-H).

Step (f): n-BuLi (10 μl, 0.03 mmol) was added to a cooled (−78° C.) solution of 4-(3-bromo-1-(4-methoxyphenyl)-1H-indol-2-yl)-3,5-dimethylisoxazole (10 mg, 0.03 mmol) under an atmosphere of nitrogen. After 5 min, 2,2,2-trifluoroacetic anhydride (7 μl, 0.05 mmol) was added. The temperature was allowed to RT and the mixture was stirred over night. 1M NaHCO₃ and DCM were added, the phases were separated and the solvents were evaporated.

Step (g): 1-(2-(3,5-dimethylisoxazol-4-yl)-1-(4-methoxyphenyl)-1H-indol-3-yl)-2,2,2-trifluoroethanone was dissolved in dry DCM and the mixture was cooled on an ice-bath under an atmosphere of nitrogen. BBr₃ (17 μl, 0.1 mmol) was added and the temperature was allowed to RT and the mixture was stirred over night. Water, DCM and some dioxane were added, the phases were separated and the solvents were evaporated. The residue was passed through a short plug of silica with EtOAc as eluent. The residue was purified by prep-HPLC to provide 1-(2-(3,5-dimethylisoxazol-4-yl)-1-(4-hydroxyphenyl)-1H-indol-3-yl)-2,2,2-trifluoroethanone in 46% yield.

Step (h): 1-(2-(3,5-dimethylisoxazol-4-yl)-1-(4-hydroxyphenyl)-1H-indol-3-yl)-2,2,2-trifluoroethanone (65 mg, 0.16 mmol), hydroxylamine hydrochloride (226 mg, 3.25 mmol) and pyridine (262 μl, 3.25) were mixed in 5 ml of 99.7% EtOH. The reaction was run at 100° C. for 20 min. The product was purified by prep-HPLC to provide 16 mg (24%) of each of the (E) and (Z) isomers of 1-(2-(3,5-dimethylisoxazol-4-yl)-1-(4-hydroxyphenyl)-1H-indol-3-yl)-2,2,2-trifluoroethanone oxime. Standard spectroscopic methods (mass spectrometry and ¹H NMR) were not conclusive as to which of Isomer A (E32) and Isomer B (E33) was the (E) or (Z) isomer.

Isomer A of 1-(2-(3,5-dimethylisoxazol-4-yl)-1-(4-hydroxyphenyl)-1H-indol-3-yl)-2,2,2-trifluoroethanone oxime (E32): ES/MS m/z: 416.23 (pos. M+H), 414.28 (neg. M−H); ¹H NMR (MeOD, 500 MHz): 7.44 (m, 1H), 7.26-7.17 (m, 3H), 7.08 (br s, 2H), 6.86 (m, 2H), 2.21 (s, 3H) and 1.89 (s, 3H).

Isomer B of 1-(2-(3,5-dimethylisoxazol-4-yl)-1-(4-hydroxyphenyl)-1H-indol-3-yl)-2,2,2-trifluoroethanone oxime (E33): ES/MS m/z: 416.24 (pos. M+H), 414.27 (neg. M−H); ¹H NMR (MeOD, 500 MHz): 7.57 (m, 1H), 7.26-7.19 (m, 3H), 7.07 (br s, 2H), 6.86 (m, 2H), 2.20 (s, 3H) and 1.92 (s, 3H).

### EXAMPLE 34

### 1-(2-(3,5-dimethylisoxazol-4-yl)-1-(4-hydroxyphenyl)-1H-indol-3-yl)ethanone oxime (E34)

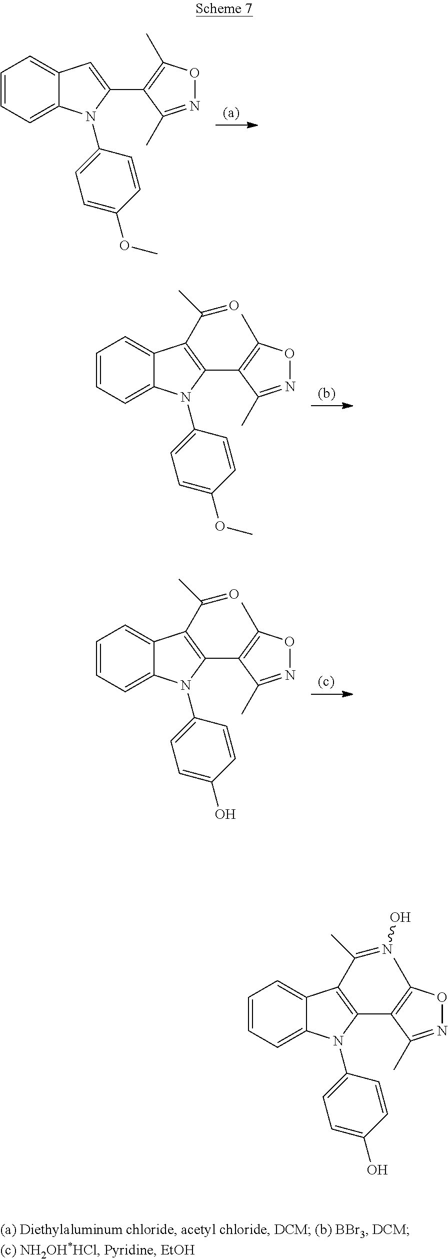

Step (a): 4-(1-(4-methoxyphenyl)-1H-indol-2-yl)-3,5-dimethylisoxazole (the intermediate product (d) from the synthesis of Examples 32 and 33) dissolved in DCM was cooled to 0° C. 1.5 eq diethylaluminum chloride was added and the reaction was stirred at 0° C. for 40 minutes. 21 eq acetyl chloride was added over five minutes and the reaction was allowed to stir at 0° C. for 2.5 hours. The reaction was quenched by slow addition of H₂O and was then diluted with DCM. The mixture was washed with H₂O and the combined aqueous layers were then extracted by DCM. The combined organic phases were evaporated to dryness in vacuo. Intermediate was purified on silica column using 80:20 n-Heptane:EtOAc as mobile phase.

Step (b): 1-(2-(3,5-dimethylisoxazol-4-yl)-1-(4-methoxyphenyl)-1H-indol-3-yl)ethanone was dissolved in DCM, cooled to 0° C. and 10 eq bortribromide were added. The mixture was stirred overnight at 5° C. MeOH was added to quench the reaction and it was then diluted with DCM. Reaction mixture was washed with brine and then the combined aqueous layers were then extracted by DCM. The combined organic phases were evaporated to dryness in vacuo. The crude product was used without further purification.

Step (c): To 1-(2-(3,5-dimethylisoxazol-4-yl)-1-(4-hydroxyphenyl)-1H-indol-3-yl)ethanone dissolved in EtOH were 10 eq hydroxylamine hydrochloride and 10 eq pyridine added. The mixture was stirred at 100° C. for 30 minutes. The solvent was evaporated and the crude product was dissolved in THF/Water-mixture and purified on reverse phase preparative HPLC. Appropriate fractions were combined and evaporated. Purity was determined by analytical HPLC. The title compound was identified by ¹H-NMR which showed that the product was a single isomer, but did not confirm whether the (E) or (Z) isomer had been obtained. ES/MS m/z: 362.25 (M+H), 360.32 (M−H); 1H NMR (methanol-d4, 500 MHz): 7.93 (m, 1H), 7-23-7.14 (m, 3H), 7.03 (m, 2H), 6.85 (m, 2H), 2.16 (s, 3H), 2.05 (s, 3H) and 1.96 (s, 3H).

### EXAMPLE 35

### 2-(3,5-dimethylisoxazol-4-yl)-1-(4-hydroxyphenyl)-1H-indole-3-carbaldehyde O-methyl oxime (E35)

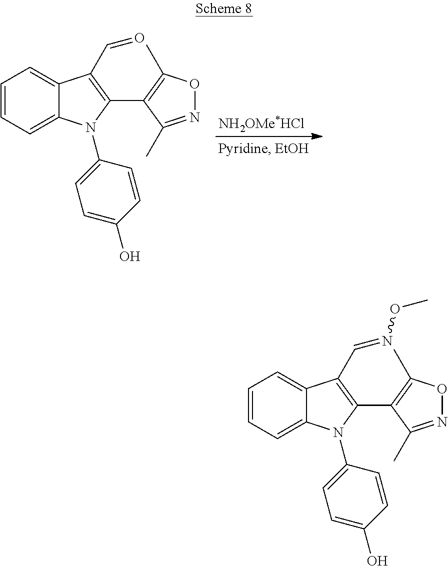

2-(3,5-Dimethylisoxazol-4-yl)-1-(4-hydroxyphenyl)-1H-indole-3-carbaldehyde (the intermediate product of step (b) from the synthesis of Example 2) was mixed with hydroxylamine and pyridine in ethanol and heated at 150° C. for 10 minutes in microwave. The reaction-mixture was purified by preparative HPLC to provide the title compound. Identification by ¹H-NMR showed that the product was a single isomer, but did not confirm whether the (E) or (Z) isomer had been obtained. ES/MS m/z: 362.2 (M+H), 360.3 (M−H); ¹H NMR (acetone-d6, 500MHz): 8.27 (m, 1H), 7.95 (s, 1H), 7.30-7.26 (m, 2H), 7.22 (m, H), 7.18 (br s, 2H), 6.96 (m, 2H), 3.93 (s, 3H), 2.28 (s, 3H) and 1.95 (s, 3H).

### EXAMPLES 36-49

The following compounds 38, 40 and 41-43 were prepared according to General Method I above. Full experimental details of the individual steps of that general method are described in Examples 1 and 2 above. The following compounds 36, 37 and 39 and 44-49 were prepared according to General Method III above, full experimental details of the individual steps of which are described in Example 31 above. If not stated otherwise, R³, R⁴, R⁵ R⁶, R⁷, R⁸, R⁹ and R¹⁰ are hydrogen.

For each of Examples 37, 40-42, 45, 47 and 48, the title compound was identified by ¹H-NMR which showed that the product was a single isomer, but did not confirm whether the (E) or (Z) isomer had been obtained. For each of Examples 38, 39, 43 and 44, the title compound was identified by ¹H-NMR which showed that the product was an approximately 1:1 mixture of the (E) and (Z) isomers. For each of Examples 36 and 46, the title compound was identified by ¹H-NMR which showed that the product was an approximately 9:1 mixture of the (E) and (Z) isomers. For Example 49, the ¹H-NMR was unclear as to whether the the product was a single isomer, or whether it was a mixture of the (E) and (Z) isomers.

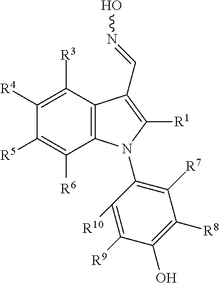

| | |
|---|---|
| E 36 | 2-(3,5-dimethylisoxazol-4-yl)-4,7-difluoro-1-(4-hydroxyphenyl)-1H-indole-3-carbaldehyde |
| | oxime |
| R¹ = 3,5-Dimethyl-isoxazol-4-yl | R³ = Fluoro |
| R⁶ = Fluoro | |

ES/MS m/z: 364.3 (pos. M + H), 362.3 (neg. M − H); ¹H NMR (methanol-d4, 500 MHz): 8.42 (s, 1H),
7.67 (m, 2H), 6.91-6.80 (m, 2H), 6.77 (m, 2H), 2.14 (s, 3H) and 2.01 (s, 3H).

| | |
|---|---|
| E 37 | 5-(4-fluoro-3-((hydroxyimino)methyl)-1-(4-hydroxyphenyl)-1H-indol-2-yl)-1-methyl-1H- |
| | pyrazole-4-carbonitrile |
| R¹ = 4-cyano-1-methyl-1H-pyrazole-5-yl | R³ = Fluoro |

ES/MS m/z: 376.2 (pos. M + H), 374.3 (neg. M − H); ¹H NMR (acetone-d6, 500 MHz): 8.58 (s, 1H),
7.83 (s, 1H), 7.31 (m, 1H), 7.19 (br s, 2H), 7.07-7.01 (m, 2H), 6.95 (br s, 2H) and 3.81 (s, 3H).

| | |
|---|---|
| E 38 | 1-(2,3-difluoro-4-hydroxyphenyl)-2-(3,5-dimethylisoxazol-4-yl)-1H-indole-3-carbaldehyde |
| | oxime |
| R¹ = 3,5-Dimethyl-isoxazol-4-yl | |
| R⁷ = Fluoro | R⁸ = Fluoro |

ES/MS m/z: 384.3 (pos. M + H), 382.2 (neg. M − H); ¹H NMR (acetone-d6, 500 MHz): 8.24 (m, 1H),
7.99 (d, 1H, J = 5.8 Hz), 7.32-7.14 (m, 4H), 7.00 (m, 1H), 2.28, 2.27 (two s, 3H), 2.03, 2.02 (two s, 3H).

| | |
|---|---|
| E 39 | 4-chloro-2-(3,5-dimethylisoxazol-4-yl)-1-(4-hydroxyphenyl)-1H-indole-3-carbaldehyde oxime |
| R¹ = 3,5-Dimethyl-isoxazol-4-yl | R³ = Cl |

ES/MS m/z: 382.2 (pos. M + H), 380.2 (neg. M − H); ¹H NMR (acetone-d6, 500 MHz): 8.97 (s, 1H),
7.26-7.13 (m, 5H), 6.95 (m, 2H), 2.16 (s, 3H) and 1.99 (s, 3H).

| | |
|---|---|
| E 40 | 1-(2-fluoro-4-hydroxyphenyl)-2-(1-methyl-1H-pyrazol-5-yl)-1H-indole-3-carbaldehyde oxime |
| R¹ = 1-methyl-1H-pyrazol-5-yl | |
| R⁷ = Fluoro | |

ES/MS m/z: 351.2 (pos. M + H), 349.1 (neg. M − H); ¹H NMR (acetone-d6, 500 MHz): 8.25 (m, 1H),
7.99 (s, 1H), 7.44 (d, 1H, J = 1.9 Hz), 7.35-7.25 (m, 3H), 7.12 (m, 1H), 6.78-6.73 (m, 2H), 6.36 (m, 1H)
and 3.63 (s, 3H).

| | |
|---|---|
| E 41 | 1-(2-fluoro-4-hydroxyphenyl)-2-(3-methylthiophen-2-yl)-1H-indole-3-carbaldehyde oxime |
| R¹ = 3-methylthiophen-2-yl | R⁷ = Fluoro |

ES/MS m/z: 367.2 (pos. M + H), 365.1 (neg. M − H); ¹H NMR (acetone-d6, 500 MHz): 8.24 (m, 1H),
8.03 (s, 1H), 7.51 (d, 1H, J = 5.0 Hz), 7.29-7.20 (m, 3H), 7.06 (m, 1H), 6.95 (d, 1H, J = 5.0 Hz), 6.75 (br s,
2H) and 1.96 (s, 3H).

| | |
|---|---|
| E 42 | 2-(3,5-dimethyl-1H-pyrazol-4-yl)-1-(2-fluoro-4-hydroxyphenyl)-1H-indole-3-carbaldehyde |
| | oxime |
| R¹ = 3,5-dimethyl-1H-pyrazol-4-yl | R⁷ = Fluoro |

ES/MS m/z: 365.2 (pos. M + H), 363.2 (neg. M − H); ¹H NMR (acetone-d6, 500 MHz): 8.21 (m, 1H),
7.94 (s, 1H), 7.24-7.19 (m, 3H), 7.07 (m, 1H), 6.77-6.71 (m, 2H), 2.06 (s, 3H) and 2.00 (s, 3H).

| | |
|---|---|
| E 43 | 1-(2-fluoro-4-hydroxyphenyl)-2-(1,3,5-trimethyl-1H-pyrazol-4-yl)-1H-indole-3-carbaldehyde |
| | oxime |
| R¹ = 1,3,5-trimethyl-1H-pyrazol-4-yl | |
| R⁷ = Fluoro | |

¹H NMR (acetone-d6, 500 MHz): 8.22 (m, 1H), 7.93, 7.92 (two s, 1H), 7.05 (m, 1H), 6.79-6.70 (m, 2H),
3.69, 3.68 (two s, 3H), 2.10, 2.03 (two s, 3H) and 1.95, 1.90 (two s, 3H). Two conformations.

| | |
|---|---|
| E 44 | 2-(3,5-dimethylisoxazol-4-yl)-4-fluoro-1-(2-fluoro-4-hydroxyphenyl)-1H-indole-3- |
| | carbaldehyde oxime |
| R¹ = 3,5-dimethylisoxazol-4-yl | R³ = 4-Fluoro |
| R⁷ = 2-Fluoro | |

ES/MS m/z: 384.1 (pos. M + H), 382.2 (neg. M − H); ¹H NMR (acetone-d6, 500 MHz): 8.46 (m, 1H),
7.37, 7.30 (two t, 1H, J = 8.9 Hz), 7.24 (m, 1H), 6.99-6.92 (m, 2H), 6.82 (m, 1H), 6.76 (m, 1H), 2.17, 2.15
(two s, 3H) and 2.02, 2.00 (two s, 314).

| | |
|---|---|
| E 45 | 5-(4-fluoro-1-(2-fluoro-4-hydroxyphenyl)-3-((hydroxyimino)methyl)-1H-indol-2-yl)-1- |
| | methyl-1H-pyrazole-4-carbonitrile |
| R¹ = 4-cyano-1-methyl-1H-pyrazole-5-yl | R³ = Fluoro |
| R⁷ = Fluoro | |

ES/MS m/z: 394.2 (pos. M + H), 392.2 (neg. M − H); ¹H NMR (acetone-d6, 500 MHz): (acetone-d6,
500 MHz): 8.59 (m, 1H), 7.85 (s, 1H), 7.48, 7.16 (two t, 1 h, J = 8.8 Hz), 7.35 (m, 1H), 7.08-7.01 (m, 2H),
6.86 (m, 1H), 6.72 (m, 1H) and 3.84, 3.77 (two s, 3H).

| | |
|---|---|
| E 46 | 6-chloro-2-(3,5-dimethylisoxazol-4-yl)-1-(4-hydroxyphenyl)-1H-indole-3-carbaldehyde oxime |
| R¹ = 3,5-dimethylisoxazol-4-yl | R⁵ = Chloro |

ES/MS m/z: 382.16 (pos. M + H), 380.16 (neg. M − H); ¹H NMR (acetone-d6, 500 MHz): 8.19 (d, 1H,
J = 8.5 Hz), 7.96 (s, 1H), 7.24-7.17 (m, 4H), 6.97 (m, 2H), 2.28 (s, 3H) and 1.97 (s, 3H).

| | |
|---|---|
| E 47 | 2-(3,5-dimethylisoxazol-4-yl)-6-fluoro-1-(4-hydroxyphenyl)-1H-indole-3-carbaldehyde oxime |
| R¹ = 3,5-dimethylisoxazol-4-yl | R⁵ = Fluoro |

ES/MS m/z: 364.3 (pos. M + H), 362.2 (neg. M − H); ¹H NMR (acetone-d6, 500 MHz): 8.20 (dd, 1H,
J = 8.8, 5.6 Hz), 7.95 (s, 1H), 7.19 (m, 2H), 7.04 (m, 1H), 6.96 (m, 2H), 6.92 (dd, 1H, J = 9.9, 2.4 Hz), 2.28
(s, 3H) and 1.96 (s, 3H).

| | |
|---|---|
| E 48 | 2-(3,5-dimethylisoxazol-4-yl)-1-(4-hydroxyphenyl)-6-(trifluoromethyl)-1H-indole-3- |
| | carbaldehyde oxime |
| R¹ = 3,5-dimethylisoxazol-4-yl | R⁵ = Trifluoromethyl |

ES/MS m/z: 416.2 (pos. M + H), 414.2 (neg. M − H); ¹H NMR (acetone-d6, 500 MHz): 8.40 (d, 1H,
J = 8.3 Hz), 8.01 (s, 1H), 7.54 (dd, 1H, J = 8.3, 1.7 Hz), 7.49 (d, 1H, J = 1.7 Hz), 7.26 (br s, 2H), 6.99 (m,
2H), 2.30 (s, 3H) and 1.99 (s, 3H).

| | |
|---|---|
| E 49 | 2-(3,5-dimethylisoxazol-4-yl)-4,6-difluoro-1-(4-hydroxyphenyl)-1H-indole-3-carbaldehyde |
| | oxime |
| R¹ = 3,5-dimethylisoxazol-4-yl | |
| R³ = Fluoro | R⁵ = Fluoro |

ES/MS m/z: 384.08 (pos. M + H), 382.15 (neg. M − H); ¹H NMR (acetone-d6, 500 MHz): 8.37 (s, 1H),
7.19 (m, 2H), 6.96 (m, 2H), 6.88 (m, 1H), 6.77 (dd, 1H, J = 9.3, 2.1 Hz), 2.18 (s, 3H) and 1.98 (s, 3H).

Description of the Estrogen Receptor Binding Assays

The estrogen receptor ligand binding assays were designed as scintillation proximity assays (SPA), employing the use of tritiated estradiol (³H-E2) and recombinant expressed biotinylated estrogen receptor binding domains. The binding domains of human ERα (ERα-LBD, pET-N-AT #1, aa 301-595) and human ERβ (ERβ-LBD, pET-N-AT #1, aa 255-530) proteins were produced in E. coli ((BL21, (DE3), pBirA)) at 22° C. in 2× LB medium, supplemented with 50 μM biotin. After 3 h of IPTG induction (0.55 mM), cells were harvested by centrifugation at 7300×g for 15 min and cell pellets stored frozen at −20° C. Extraction of ERα-LBD and ERβ-LBD proteins was performed using 5 g of cells suspended in 50 mL of extraction buffer (50 mM Tris, pH 8.0, 100 mM KCl, 4 mM EDTA, 4 mM DDT and 0.1 mM PMSF). The cell suspension was run twice through a Microfluidizer M-110L (Microfluidics) and centrifuged at 15,000×g for 60 min. The supernatant was aliquoted and stored at −70° C.

ERα-LBD or ERβ-LBD extracts were diluted in assay buffer (18 mM K₂HPO₄, 2 mM KH₂PO₄, 20 mM Na₂MoO₄, 1 mM EDTA, 1 mM TCEP) 1:676 and 1:517 for alpha and beta respectively. The diluted receptor extracts had a receptor concentration of about 900 fmol/L. The extracts were then preincubated with streptavidin-coated polyvinyltoluene SPA beads (RPNQ0007, GE Healthcare) at a concentration of 0.43 mg/mL for 1 hr at room temperature.

Test compounds were evaluated over a range of concentrations, typically from 157 μM to 37.5 pM. The test compound stock solutions were prepared in 100% DMSO at 5 fold the final concentration intended for testing in the assay. The amount of DMSO in the test wells of the 384 well plates was thus 20%. Aliquots (18 μL/well) of test compounds were transferred to the assay plates, followed by 35 μL/well of the preincubated receptor/SPA bead mix and finally 35 μL/well of 3 nM ³H-E2. The plates were covered with plastic sealers, centrifuged for 1 minute at 1000 rpm, and equilibrated over night on a shaker at room temperature. Finally, the plates were centrifuged for 5 minutes at 2000 rpm and analysed on a plate scintillation counter (e. g. a PerkinElmer Microbeta 1450 Trilux).

For compounds able to displace ³H-E2 from the receptor, an IC₅₀-value (the concentration required to inhibit 50% of the binding of ³H-E2) was determined by a non-linear, four parameter logistic model; b=((bmax−bmin)/(1+(I/IC₅₀)S))+bmin. Here, bmax and bmin are maximum and minimum plateaus of the fitted curve, I represents the concentration of binding inhibitor, IC₅₀ is the concentration of inhibitor at half maximal binding, and S is a slope factor. The Microbeta-instrument engaged in these experiments corrected for individual variations between the detectors and thus presented the signal as corrected counts per minute (ccpm).

Transactivation Assay 1: Transactivation Assay in Human Embryonic Kidney 293 Cells Stably Transfected with pERE-ALP and Human Estrogen Receptor Alpha

The expression vector pMThERα contains an insert of wild type human estrogen receptor alpha with deleted leader. The pERE-ALP reporter construct contains the gene for the secreted form of placental alkaline phosphatase (ALP) and the vitellogenin estrogen response element (ERE). The human embryonic kidney 293 cells are transfected in two steps. Firstly, a stable clone mix transfected with the pERE-ALP reporter gene construct and pSV2-Neo for selection is developed. Secondly, the stable clone mix is transfected with pMThERα and a pKSV-Hyg resistance vector for selection. All transfections are performed using Lipofectamine (Invitrogen) according to supplier's recommendations. A selected clone with both pERE-ALP and pMThERα is used for the transactivation assay.

The cells are seeded in 384-well plates at 12 500 cells per well in Ham's F12 Coon's modification (without phenol red) with 10% dextran-coated charcoal treated (DCC) fetal bovine serum (FBS), 2 mM L-glutamine and 50 μg/mlgentamicin. After 24 h incubation (37° C., 5% CO₂) the seeding medium is discarded and replaced with 20 μl Ham's F12 Coon's modification (without phenol red) with 1.5% DCC-FCS, 2 mM L-glutamine and supplemented with 100 U/ml penicillin and 100 μg/ml streptomycin. The selected compounds are added to the wells in 12 concentrations ranging from 3.3 pM to 33 μM. The compounds are dissolved in 100% dimethylsulphoxide (DMSO) and the final concentration of DMSO in the assay is 0.1%. After 72 h incubation (37° C., 5% CO₂) the medium is assayed for ALP activity by a chemiluminescence assay; a 10 μl aliquot of the cell culture medium is mixed with 100 μl assay buffer (0.1 M diethanolamine, 1 mM MgCl₂) and 0.5 mM disodium 3-(4-methoxyspiro1,2-dioxetane-3,2′-(5′-chloro)-tricyclo[3.3.1.13,7]decan-4-yl)phenyl phosphate (CSPD) (Tropix, Applied Biosystems) and incubated for 20 min at 37° C. and 15 min at room temperature before measurement chemiluminescent light signal (one second per well) in a Wallac Microbeta Trilux 1450-028 (PerkinElmer). The half maximal effective concentrations (EC₅₀) are calculated from the curves fitted to the concentration-response data with a four parameter logistic model in XLfit software version 2.0 (IDBS) or later.

Transactivation Assay 2: Transactivation Assay in Human Embryonic Kidney 293 Cells Stably Transfected with pERE2-ALP and Human Estrogen Receptor Beta

Generation of stable HEK293 cell lines (CRL-1573; American Type Culture Collection) expressing the reporter vector pERE2-ALP and human estrogen receptor beta (hERβ 530) have been described (Mol Pharmacol 1998, 54,105-112; Endocrinology 2002, 143, 1558-1561).

The cells were seeded in 384-well plates at 12 500 cells per well in Ham's F12 Coon's modification (without phenol red) with 10% dextran-coated charcoal treated (DCC) fetal bovine serum (FBS), 2 mM L-glutamine and 50 μg/ml gentamicin. After 24 h incubation (37° C., 5% CO₂) the seeding medium was discarded and replaced with 20 μl Ham's F12 Coon's modification (without phenol red) with 1.5 DCC-FCS, 2 mM L-glutamine and supplemented with 100 U/ml penicillin and 100 μg/ml streptomycin. The selected compounds were added to the wells in 12 concentrations ranging from 3.3 pM to 33 μM. The compounds were dissolved in 100% dimethylsulfoxide (DMSO) and the final concentration of DMSO in the assay was 0.1%. After 72 h incubation (37° C., 5% CO₂) the medium was assayed for ALP activity by a chemiluminescence assay; a 10 μl aliquot of the conditioned medium was mixed with 100 μl assay buffer (0.1 M diethanolamine, 1 mM MgCl₂) and 0.5 mM disodium 3-(4-methoxyspiro 1,2-dioxetane-3,2′-(5′-chloro)-tricyclo[3.3.1.13,7]decan-4-yl)phenyl phosphate (CSPD) (Tropix, Applied Biosystems) and incubated for 20 min at 37° C. and 15 min at room temperature before measurement of the chemiluminescent signal (one second per well) in a Wallac Microbeta Trilux 1450-028 (PerkinElmer). The ALP activity expressed in LCPS is directly proportional to the level of ALP expressed by the cells. The half maximal effective concentrations of the test compounds (EC50) were calculated from the curves fitted to the concentration-response data with a four parameter logistic model in XLfit software version 2.0 (IDBS) or later.

The compounds of Examples 1-49 exhibit binding affinities to the estrogen receptor α-subtype in the range of IC₅₀ 1 to 10,000 nM or to the estrogen receptor β-subtype in the range of IC₅₀ 1 to 10,000 nM.

The compounds of Examples 1-49 exhibit a potency in the range of EC₅₀ 1 to 10,000 nM at the estrogen receptor α-subtype in transactivation assay 1 and a potency in the range of EC₅₀ 1 to 10,000 nM at the estrogen receptor β-subtype in transactivation assay 2.

Preferred Example compounds of the invention are those which exhibit a binding affinity to the estrogen receptor β-subtype at lower concentrations within the IC₅₀ range shown above. For example, the compounds of Examples 1, 2, 5-8, 10, 13, 16, 19, 21-24, 27-29, 31, 33, 40, 41 and 46-49 exhibit a binding affinity to the estrogen receptor β-subtype in the range of IC₅₀ 1 to 50 nM in the binding assay.

Preferred Example compounds of the invention are those which are selective for the estrogen receptor β-subtype over the estrogen receptor α-subtype in the binding assay. For example, the compounds of Examples 2, 13, 16, 21, 22, 25, 28, 29, 34, 38, 43, 44 and 46-49 display selectivity for the estrogen receptor β-subtype of 50 or greater in the binding assay.

Preferred Example compounds of the invention are those which display a potency at the estrogen receptor β-subtype at lower concentrations within the EC₅₀ range shown above. For example, the compounds of Examples 1, 2, 5-8, 10, 13, 16, 21, 22, 24, 27-30, 31, 38, 40, 41, 43, 46 and 47 exhibit a potency in the range of EC₅₀ 1 to 10 nM at the estrogen receptor β-subtype in transactivation assay 2.

Preferred Example compounds of the invention are those which are selective for the estrogen receptor β-subtype over the estrogen receptor α-subtype in the transactivation assays 1 and 2. For example, the compounds of Examples 2, 15, 16, 21, 27, 34, 43 and 47 display selectivity for the estrogen receptor β-subtype of 50 or greater in the transactivation assays.

Particularly preferred Example compounds of the invention are those which exhibit both a binding affinity to the estrogen receptor β-subtype at lower concentrations within the IC₅₀ range shown above and a potency at the estrogen receptor β-subtype at lower concentrations within the EC₅₀ range shown above. For example, the compounds of Examples 1, 2, 5-8, 10, 13, 16, 21, 22, 24, 27-29, 31, 38, 40, 41, 46 and 47 exhibit a binding affinity to the estrogen receptor β-subtype in the range of IC₅₀ 1 to 50 nM in binding assay 1 and a potency in the range of EC₅₀ 1 to 10 nM at the estrogen receptor β-subtype in transactivation assay 2.

1. A compound of formula (I) or a pharmaceutically acceptable ester, amide, solvate or salt thereof, including a salt of such an ester or amide, and a solvate of such an ester, amide or salt, 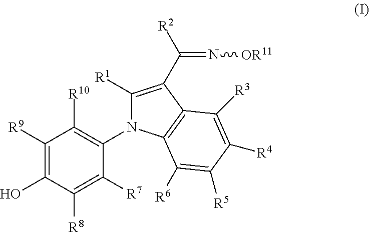
wherein R¹ is selected from the group consisting of C₃₋₈cycloalkyl, phenyl, and 5-10 membered heterocyclyl, wherein said phenyl or heterocyclyl group can be either unsubstituted or substituted with from 1 to 3 substituents, each substituent being selected from the group consisting of OR^{A}, halogen, cyano, nitro, —C(O)C₁₋₄alkyl, C₁₋₆alkyl, C₂₋₆alkenyl, C₂₋₆alkynyl, haloC₁₋₆ alkyl, dihaloC₁₋₆alkyl and trihaloC₁₋₆alkyl;
R² is selected from the group consisting of hydrogen, C₁₋₄alkyl, haloC₁₋₄alkyl, dihaloC₁₋₄alkyl, trihaloC₁₋₄alkyl, C₂₋₄alkenyl, and C₂₋₄alkynyl;
each of R³, R⁴, R⁵, R⁶, R⁷, R⁸, R 9 R , R⁹ and R¹⁰ is independently selected from the group consisting of hydrogen, OR^{A}, halogen, cyano, nitro, C₁₋₆alkyl, C₂₋₆alkenyl, C₂₋₆alkynyl, haloC₁₋₆alkyl, dihaloC₁₋₆alkyl and trihaloC₁₋₆alkyl;
each R^{A} is independently selected from the group consisting of hydrogen, C₁₋₆alkyl, C₂₋₆alkenyl, C₂₋₆alkynyl, C₃₋₈cycloalkyl, C₃₋₈cycloalkylC₁₋₆alkyl C₆₋₁₀aryl and C₆₋₁₀arylC₁₋₆alkyl, each optionally substituted by from 1 to 3 halogen atoms; and
R¹¹ is selected from hydrogen and methyl;
with the proviso that when R¹ is a 5-membered heterocyclyl, and each of R², R³, R⁴, R⁶ and R¹¹ is hydrogen, then R⁵ is not methoxy.

2. A compound as claimed in claim 1, in which each R^{A} is independently selected from the group consisting of hydrogen, C₁₋₄alkyl, C₂₋₄alkenyl, C₂₋₄alkynyl, C₃₋₆cycloalkyl, phenyl and benzyl.

3. A compound as claimed in claim 2, in which each R^{A} independently represents hydrogen or C₁₋₄alkyl.

4. A compound as claimed in claim 1, in which R¹ is selected from the group consisting of phenyl and 5-6 membered heterocyclyl, wherein said phenyl or heterocyclyl group can either be unsubstituted or substituted by 1 to 3 substituents selected from the group consisting of OR^{A}, halogen, cyano, —C(O)C₁₋₄alkyl, C₁₋₄alkyl, C₂₋₄alkenyl, C₂₋₄alkynyl, haloC₁₋₄alkyl, dihaloC₁₋₄alkyl and trihaloC₁₋₄alkyl.

5. A compound as claimed in claim 4, in which R¹ is selected from the group consisting of phenyl and 5-membered heterocyclyl, wherein said phenyl or heterocyclyl group can either be unsubstituted or substituted by 1 to 3 substituents selected from halogen, cyano, C₁₋₄alkyl, —C(O)C₁₋₄alkyl, and OR^{A} in which R^{A} represents hydrogen or C₁₋₄alkyl.

6. A compound as claimed in claim 1, in which R² is selected from the group consisting of hydrogen, C₁₋₄alkyl, C₂₋₄alkenyl, C₂₋₄alkynyl and trihaloC₁₋₄alkyl.

7. A compound as claimed in claim 6, in which R² is selected from the group consisting of hydrogen and C₁₋₄alkyl.

8. A compound as claimed in claim 1, in which each of R³, R⁴, R⁵, R⁶, R⁷, R⁸, R⁹ and R¹⁰ is independently selected from the group consisting of hydrogen, OR^{A}, halogen, cyano, C₁₋₄alkyl, haloC₁₋₄alkyl, dihaloC₁₋₄alkyl, and trihaloC₁₋₄alkyl.

9. A compound as claimed in claim 8, in which each of R³, R⁴, R⁵, R⁶, R⁷, R⁸, R⁹ and R¹⁰ is independently selected from the group consisting of hydrogen, OH, halogen, cyano, methyl and trifluoromethyl.

10. A compound as claimed in claim 1, in which R¹ is selected from the group consisting of phenyl and 5-6 membered heterocyclyl, wherein said phenyl or heterocyclyl group may be either unsubstituted or substituted by 1 to 3 substituents selected from the group consisting of OR^{A}, halogen, cyano, C(O)C₁₋₄alkyl, C₁₋₄alkyl, C₂₋₄alkenyl, C₂₋₄alkynyl, and R² is selected from the group consisting of hydrogen, C₁₋₄alkyl, C₂₋₄alkenyl, C₂₋₄alkynyl and trihaloC₁₋₄alkyl; each of R³, R⁴, R⁵, R⁶, R⁷, R⁸, R⁹ and R¹⁰ is independently selected from the group consisting of hydrogen, OR^{A}, halogen, cyano, C₁₋₄alkyl, haloC₁₋₄alkyl, dihaloC₁₋₄alkyl, and trihaloC₁₋₄alkyl; each R^{A} is independently selected from the group consisting of hydrogen, C₁₋₄alkyl, C₂₋₄alkenyl, C₂₋₄alkynyl, C₃₋₆cycloalkyl, phenyl and benzyl; and R¹¹ is hydrogen.

11. A compound as claimed in claim 10, in which R¹ is selected from the group consisting of phenyl and 5-membered heterocyclyl, wherein said phenyl or heterocyclyl group can either be unsubstituted or substituted by 1 to 3 substituents selected from halogen, cyano, C₁₋₄alkyl, —C(O)C₁₋₄alkyl, and OR^{A}; and each R^{A} independently represents hydrogen or C₁₋₄alkyl.

12. A compound as claimed in claim 10, in which R² is selected from the group consisting of hydrogen, C₁₋₄alkyl and trihaloC₁₋₄alkyl.

13. A compound as claimed in claim 10, in which each of R³, R⁴, R⁵, R⁶, R⁷, R⁸, R⁹ and R¹⁰ is independently selected from the group consisting of hydrogen, OH, halogen, cyano, methyl, or trifluoromethyl.

14. A compound as claimed in claim 1, which is any one of the following compounds: 1-(4-Hydroxy-phenyl)-2-pyrrol-1-yl-1H-indole-3-carbaldehyde oxime (E1);
2-(3,5-Dimethyl-isoxazol-4-yl)-1-(4-hydroxy-phenyl)-1H-indole-3-carbaldehyde oxime (E2);
1-(4-Hydroxy-phenyl)-2-(1H-pyrazol-3-yl)-1H-indole-3-carbaldehyde oxime (E3);
1-(4-Hydroxy-phenyl)-2-(3-methyl-3H-imidazol-4-yl)-1H-indole-3-carbaldehyde oxime (E4); 1-(4-Hydroxy-phenyl)-2-phenyl-1H-indole-3-carbaldehyde oxime (E5);
1-(4-Hydroxy-phenyl)-1H-indole-3-carbonitrile (E6);
1-(4-Hydroxy-phenyl)-2-thiophen-3-yl-1H-indole-3-carbaldehyde oxime (E7);
1-(4-Hydroxy-phenyl)-2-(3-methyl-thiophen-2-yl)-1H-indole-3-carbaldehyde oxime (E8);
1-[1-(4-Hydroxy-phenyl)-2-phenyl-1H-indol-3-yl]-ethanone oxime (E9);
1-(4-Hydroxy-phenyl)-2-(4-methyl-thiophen-3-yl)-1H-indole-3-carbaldehyde oxime (E10);
2-(3,5-Dimethyl-1H-pyrazol-4-yl)-1-(4-hydiOxy-phenyl)-1H-indole-3-carbaldehyde oxime (E11);
1-(4-Hydroxy-phenyl)-2-(5-methyl-1H-pyrazol-4-yl)-1H-indole-3-carbaldehyde oxime (E12);
1-(4-Hydroxy-phenyl)-2-(2-methyl-2H-pyrazol-3-yl)-1H-indole-3-carbaldehyde oxime (E13);
2-[3-(Hydroxyimino-methyl)-1-(4-hydroxy-phenyl)-1H-indol-2-yl]-furan-3-carbonitrile (E14);
2-[3-(Hydroxyimino-methyl)-1-(4-hydroxy-phenyl)-1H-indol-2-yl]-thiophene-3-carbonitrile (E15);
5-[3-(Hydroxyimino-methyl)-1-(4-hydroxy-phenyl)-1H-indol-2-yl]-1-methyl-1H-pyrazole-4-carbonitrile (E16);
2-[3-(Hydroxyimino-methyl)-1-(4-hydroxy-phenyl)-1H-indol-2-yl]-benzonitrile (E17);
1-Ethyl-2-[3-(hydroxyimino-methyl)-1-(4-hydroxy-phenyl)-1H-indol-2-yl]-1H-pyrrole-3-carbonitrile (E18);
4-Fluoro-1-(4-hydroxy-phenyl)-2-pyrrol-1-yl-1H-indole-3-carbaldehyde oxime (E19);
4-Fluoro-1-(4-hydroxy-phenyl)-2-phenyl-1H-indole-3-carbaldehyde oxime (E20);
2-(3,5-Dimethyl-isoxazol-4-yl)-7-fluoro-1-(4-hydroxy-phenyl)-1H-indole-3-carbaldehyde oxime (E21);
2-(3,5-Dimethyl-isoxazol-4-yl)-5-fluoro-1-(4-hydroxy-phenyl)-1H-indole-3-carbaldehyde oxime (E22);
5-Fluoro-1-(4-hydroxy-phenyl)-2-phenyl-1H-indole-3-carbaldehyde oxime (E23);
5-Fluoro-1-(4-hydroxy-phenyl)-2-pyrrol-1-yl-1H-indole-3-carbaldehyde oxime (E24);
2-(3,5-Dimethyl-isoxazol-4-yl)-4-fluoro-1-(4-hydroxy-phenyl)-1H-indole-3-carbaldehyde oxime (E25);
1-(3-Chloro-4-hydroxy-phenyl)-2-(3,5-dimethyl-isoxazol-4-yl)-1H-indole-3-carbaldehyde oxime (E26);
2-(3,5-Dimethyl-isoxazol-4-yl)-1-(2-fluoro-4-hydroxy-phenyl)-1H-indole-3-carbaldehyde oxime (E27);
1-(2,5-Difluoro-4-hydroxy-phenyl)-2-(3,5-dimethyl-isoxazol-4-yl)-1H-indole-3-carbaldehyde oxime (E28);
2-(3,5-Dimethyl-isoxazol-4-yl)-1-(3-fluoro-4-hydroxy-phenyl)-1H-indole-3-carbaldehyde oxime (E29);
1-(3,5-Difluoro-4-hydroxy-phenyl)-2-(3,5-dimethyl-isoxazol-4-yl)-1H-indole-3-carbaldehyde oxime (E30);
2-(3,5-dimethylisoxazol-4-yl)-5,6-difluoro-1-(4-hydroxyphenyl)-1H-indole-3-carbaldehyde oxime (E31);
(E)-1-(2-(3,5-dimethylisoxazol-4-yl)-1-(4-hydroxyphenyl)-1H-indol-3-yl)-2,2,2-trifluoroethanone oxime (E32);
(Z)-1-(2-(3,5-dimethylisoxazol-4-yl)-1-(4-hydroxyphenyl)-1H-indol-3-yl)-2,2,2-trifluoroethanone oxime (E33);
1-(2-(3,5-dimethylisoxazol-4-yl)-1-(4-hydroxyphenyl)-1H-indol-3-yl)ethanone oxime (E34);
2-(3,5-dimethylisoxazol-4-yl)-1-(4-hydroxyphenyl)-1H-indole-3-carbaldehyde O-methyl oxime (E35);
2-(3,5-dimethylisoxazol-4-yl)-4,7-difluoro-1-(4-hydroxyphenyl)-1H-indole-3-carbaldehyde oxime (E36);
5-(4-fluoro-3-((hydroxyimino)methyl)-1-(4-hydroxyphenyl)-1H-indol-2-yl)-1-methyl-1H-pyrazole-4-carbonitrile (E37);
1-(2,3-difluoro-4-hydroxyphenyl)-2-(3,5-dimethylisoxazol-4-yl)-1H-indole-3-carbaldehyde oxime (E38);
4-chloro-2-(3,5-dimethylisoxazol-4-yl)-1-(4-hydroxyphenyl)-1H-indole-3-carbaldehyde oxime (E39);
1-(2-fluoro-4-hydroxyphenyl)-2-(1-methyl-1H-pyrazol-5-yl)-1H-indole-3-carbaldehyde oxime (E40);
1-(2-fluoro-4-hydroxyphenyl)-2-(3-methylthiophen-2-yl)-1H-indole-3-carbaldehyde oxime (E41);
2-(3,5-dimethyl-1H-pyrazol-4-yl)-1-(2-fluoro-4-hydroxyphenyl)-1H-indole-3-carbaldehyde oxime (E42);
1-(2-fluoro-4-hydroxyphenyl)-2-(1,3,5-trimethyl-1H-pyrazol-4-yl)-1H-indole-3-carbaldehyde oxime (E43);
2-(3,5-dimethylisoxazol-4-yl)-4-fluoro-1-(2-fluoro-4-hydroxyphenyl)-1H-indole-3-carbaldehyde oxime (E44);
5-(4-fluoro-1-(2-fluoro-4-hydroxyphenyl)-3-((hydroxyimino)methyl)-1H-indol-2-yl)-1-methyl-1H- pyrazole-4-carbonitrile (E45);
6-chloro-2-(3,5-dimethylisoxazol-4-yl)-1-(4-hydroxyphenyl)-1H-indole-3-carbaldehyde oxime (E46);
2-(3,5-dimethylisoxazol-4-yl)-6-fluoro-1-(4-hydroxyphenyl)-1H-indole-3-carbaldehyde oxime (E47);
2-(3,5-dimethylisoxazol-4-yl)-1-(4-hydroxyphenyl)-6-(trifluoromethyl)-1H-indole-3-carbaldehyde oxime (E48);
2-(3,5-dimethylisoxazol-4-yl)-4,6-difluoro-1-(4-hydroxyphenyl)-1H-indole-3-carbaldehyde oxime (E49);
or a pharmaceutically acceptable ester, amide, solvate or salt thereof, including a salt of such an ester or amide, and a solvate of such an ester, amide or salt thereof

15. A pharmaceutical composition which comprises a compound as claimed in claim 1, together with a pharmaceutically acceptable carrier.

16. A pharmaceutical composition as claimed in claim 15 further comprising an additional therapeutic agent selected from: an organic bisphosphonate; a cathepsin K inhibitor; an estrogen; an estrogen receptor modulator; an androgen receptor modulator; an inhibitor of osteoclast proton ATPase; an inhibitor of HMG-CoA reductase; an integrin receptor antagonist; anosteoblast anabolic agent; calcitonin; Vitamin D; a synthetic Vitamin D analogue; an anti-depressant; an anxiolytic; an anti-psychotic; an anti-cancer agent; or a pharmaceutically acceptable salt thereof or a mixture thereof.

17. A compound as claimed in claim 1, for use as a medicament.

18. A compound as claimed in claim 17, for, use in the treatment or prophylaxis of a condition associated with a disease or disorder associated with estrogen receptor activity.

19. (canceled)

20. A method for the treatment or prophylaxis of a disease or disorder associated with estrogen receptor activity in a mammal, which comprises administering to the mammal a therapeutically effective amount of a compound as claimed in claim 1.

21. (canceled)

22. The method of claim 20, wherein the condition associated with a disease or disorder associated with estrogen receptor activity is selected from bone loss, bone fractures, osteoporosis, cartilage degeneration, endometriosis, uterine fibroid disease, hot flashes, increased levels of LDL cholesterol, cardiovascular disease, impairment of cognitive functioning, age-related mild cognitive impairment, cerebral degenerative disorders, restenosis, gynecomastia, vascular smooth muscle cell proliferation, obesity, incontinence, anxiety, depression, perimenopausal depression, post-partum depression, premenstrual syndrome, manic depression, dementia, obsessive compulsive behavior, attention deficit disorder, attention deficit hyperactivity disorder, sleep disorders, irritability, impulsivity, anger management, hearing disorders, multiple sclerosis, Parkinson's disease, Alzheimer's disease, Huntington's disease, amyotrophic lateral sclerosis, spinal cord injury, stroke, autoimmune disease, inflammation, IBD, IBS, sexual dysfunction, hypertension, retinal degeneration, lung cancer, colon cancer, breast cancer, uterus cancer, prostate cancer and cholangiocarcinoma.
